SEQUENCE LISTING (17 sequences)

SEQ ID NO: 1 - DNA, 502 bp, Homo sapiens
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggga

SEQ ID NO: 2 - DNA, 917 bp, Homo sapiens
aggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggg

SEQ ID NO: 3 - DNA, 179 bp, Homo sapiens
gagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 4 - DNA, 254 bp, Homo sapiens
aggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaa

SEQ ID NO: 5 - DNA, 254 bp, Homo sapiens
aggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatg

SEQ ID NO: 6 - DNA, 253 bp, Homo sapiens
tgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcct

SEQ ID NO: 7 - DNA, 231 bp, Homo sapiens
caccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggg

SEQ ID NO: 8 - DNA, 681 bp, Artificial Sequence
  Synthetic Construct
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 9 - DNA, 2209 bp, Homo sapiens
gagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 10 - protein, 354 aa, Homo sapiens
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRSHRSDGEFSPHSHYSDSDEAS

SEQ ID NO: 11 - DNA, 1062 bp, Homo sapiens
atgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagt

SEQ ID NO: 12 - protein, 351 aa, Mus musculus
MSRLLHAEEWAEVKELGDHHRHPQPHHVPPLTPQPPATLQARDLPVYPAELSLLDSTDPRAWLTPTLQGLCTARAAQYLLHSPELGASEAAAPRDEADSQGELVRRSGCGGLSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPNVGEQPPPPTASCKNDHHHLRTASSYEGGAGASAVAGAQPAPGGGPRPTPPGPCRTRFSGPASSGGYSVQLDALHFPAFEDRALTAMMAQKDLSPSLPGGILQPVQEDNSKTSPRSHRSDGEFSPHSHYSDSDEAS

SEQ ID NO: 13 - DNA, 1053 bp, Mus musculus
atgtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaggagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagt

SEQ ID NO: 14 - DNA, 6988 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccacaccggtgaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcccgcggccgcgccaccatgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagtacgcgtggaagcggagctactaacttcagcctgctgaagcaggctggcgacgtggaggagaaccctggacctatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 15 - DNA, 5751 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccacaccggtgaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcccgcggccgcgccaccatgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagttagaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 16 - DNA, 548 bp, Artificial Sequence
  Synthetic Construct
gatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcga

SEQ ID NO: 17 - DNA, 425 bp, Artificial Sequence
  Synthetic Construct
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa

(19) United States
(12) Patent Application Publication
(10) Pub. No.: US 2023/0181767 A1
(43) Pub. Date: Jun. 15, 2023

(54) COMPOSITIONS AND METHODS FOR PROMOTING HAIR CELL REGENERATION

(71) Applicant: Decibel Therapeutics, Inc., Boston, MA (US)

(72) Inventors: Joseph BURNS, Newton, MA (US); Tyler GIBSON, Boston, MA (US); Ryan MCCARTHY, Ashland, MA (US); Gabriela PREGERNIG, Cambridge, MA (US); Tian YANG, Boston, MA (US)

(21) Appl. No.: 17/924,578

(22) PCT Filed: May 14, 2021
(86) PCT No.: PCT/US2021/032480

§ 371 Date: Nov. 10, 2022

Related U.S. Application Data
(60) Provisional application No. 63/109,420, filed on Nov. 4, 2020.
(60) Provisional application No. 63/025,039, filed on May 14, 2020.

(51) Int. Cl. A61K 48/00; A61P 27/16

(52) U.S. Cl. CPC A61K 48/0058; A61K 48/0033; A61P 27/16

(57) ABSTRACT

The disclosure provides nucleic acidvectors containing a high expression promoter, such as a high expression, supporting cell-specific promoter, operably linked to a polynucleotide encoding Atoh1. Such vectors and compositions containing the same can be used to induce robust regeneration of mature hair cells (e.g., cochlear and/or vestibular hair cell regeneration). Accordingly, the nucleic acid vectors and compositions described herein can be used to treat subjects having or at risk of developing hearing loss or vestibular dysfunction.

### SEQUENCE LISTING

The instant application contains a Sequence Listing which has been submitted electronically in ASCII format and is hereby incorporated by reference in its entirety. The ASCII copy, created on May 13, 2021, is named 51124-085WO3_Sequence_Listing_5_13_21_ST25 and is 36,040 bytes in size.

### BACKGROUND

Hearing loss is a major public health issue that is estimated to affect nearly 15% of school-age children and one out of three people by age sixty-five. The most common type of hearing loss is sensorineural hearing loss, a type of hearing loss caused by defects in the cells of the inner ear, such as cochlear hair cells, or the neural pathways that project from the inner ear to the brain. Sensorineural hearing loss is often acquired, and has a variety of causes, including acoustic trauma, disease or infection, head trauma, ototoxic drugs, and aging. There are also genetic causes of sensorineural hearing loss, such as mutations in genes involved in the development and function of cells of the inner ear. Mutations in over 90 such genes have been identified, including mutations inherited in an autosomal recessive, autosomal dominant, or X-linked pattern.

Factors that disrupt the development, survival, or integrity of cochlear hair cells, such as genetic mutations, disease or infection, ototoxic drugs, head trauma, and aging, may similarly affect vestibular hair cells and are, therefore, also implicated in vestibular dysfunction, including vertigo, dizziness, and imbalance. Indeed, patients carrying mutations that disrupt hair cell development or function can present with both hearing loss and vestibular dysfunction, or either disorder alone. Approximately 35% of US adults age 40 years and older exhibit balance disorders and this proportion dramatically increases with age, leading to disruption of daily activities, decline in mood and cognition, and an increased prevalence of falls among the elderly. Effective treatment for hearing loss or vestibular dysfunction in patients who have experienced damage to or loss of hair cells will require significant hair cell regeneration. Accordingly, there is a need for new therapeutic approaches that can induce robust hair cell regeneration in the auditory and/or vestibular system.

### SUMMARY OF THE INVENTION

The invention provides compositions and methods for promoting the expression of atonal BHLH transcription factor 1 (Atoh1) in supporting cells of the inner ear (e.g., cochlear and/or vestibular supporting cells). The compositions and methods described herein relate to nucleic acid vectors (e.g., adeno-associated virus (AAV) vectors) containing a high expression promoter, such as a high expression, supporting cell-specific promoter, operably linked to a polynucleotide encoding Atoh1. The nucleic acid vectors described herein can be used to induce or increase hair cell regeneration (e.g., cochlear and/or vestibular hair cell regeneration) and to treat hearing loss and/or vestibular dysfunction in a subject in need thereof (e.g., a human subject).

In a first aspect, the invention provides a nucleic acid vector including a high expression supporting cell-specific promoter operably linked to a polynucleotide encoding Atoh1.

In some embodiments, the high expression supporting cell-specific promoter is a GFAP promoter having the sequence of formula A-B-C, wherein A has the sequence of SEQ ID NO: 1 (positions −1757 to −1256 relative to the transcriptional start site of the human GFAP gene), B has the sequence of SEQ ID NO: 2 (positions −1050 to −133 relative to the transcriptional start site of the human GFAP gene), and C has the sequence of SEQ ID NO: 3 (positions −132 to +47 relative to the transcriptional start site of the human GFAP gene), in which all or part of B is optionally absent. In some embodiments, nucleotides 1-254 of B (positions −1050 to −797 relative to the transcriptional start site of the human GFAP gene, corresponding to SEQ ID NO: 4) are present. In some embodiments, nucleotides 230-483 of B (positions −821 to −568 relative to the transcriptional start site of the human GFAP gene, corresponding to SEQ ID NO: 5) are present. In some embodiments, nucleotides 459-711 of B (positions −592 to −339 relative to the transcriptional start site of the human GFAP gene, corresponding to SEQ ID NO: 6) are present. In some embodiments, nucleotides 687-917 of B (positions −363 to −133 relative to the transcriptional start site of the human GFAP gene, corresponding to SEQ ID NO: 7) are present. In some embodiments, all of B is present.

In some embodiments, the high expression supporting cell-specific promoter has the sequence of SEQ ID NO: 8.

In some embodiments, the polynucleotide encoding Atoh1 encodes an Atoh1 polypeptide having the sequence of SEQ ID NO: 10 or a variant thereof having one or more (e.g., 1, 2, 3, 4, 5, 6, 7, 8, 9, 10, 11, 12, 13, 14, 15, 16, 17, 18, 19, 20 or more) conservative amino acid substitutions. In some embodiments, no more than 10% (10%, 9%, 8%, 7%, 6%, 5%, 4%, 3%, 2%, 1%, or fewer) of the amino acids in the Atoh1 polypeptide variant are conservative amino acid substitutions. In some embodiments, the polynucleotide encoding Atoh1 encodes an Atoh1 polypeptide having the sequence of SEQ ID NO: 10. In some embodiments, the polynucleotide encoding Atoh1 has the sequence of SEQ ID NO: 11.

In some embodiments, the nucleic acid vector further includes inverted terminal repeat sequences (ITRs). In some embodiments, the nucleic acid vector includes a first ITR sequence 5′ of the promoter and a second ITR sequence 3′ of the polynucleotide encoding Atoh1. In some embodiments, the ITRs are AAV2 ITRs. In some embodiments, the ITRs have at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to AAV2 ITRs.

In some embodiments, the nucleic acid vector further includes a polyadenylation (poly(A)) sequence. In some embodiments, the poly(A) sequence is a bovine growth hormone (bGH) poly(A) sequence. In some embodiments, the poly(A) sequence is positioned 3′ of the polynucleotide encoding Atoh1. In embodiments in which the nucleic acid vector includes first and second ITR sequences, the poly(A) sequence is positioned 3′ of the polynucleotide encoding Atoh1 and 5′ of the second ITR sequence.

In some embodiments, the nucleic acid vector further includes a Woodchuck Posttranscriptional Regulatory Element (WPRE). In some embodiments, the WPRE has the sequence of SEQ ID NO: 16 or SEQ ID NO: 17. In some embodiments, the WPRE is positioned 3′ of the polynucleotide encoding Atoh1. In embodiments in which the nucleic acid vector includes a poly(A) sequence, the WPRE is positioned 3′ of the polynucleotide encoding Atoh1 and 5′ of the poly(A) sequence.

In some embodiments, the nucleic acid vector contains a polynucleotide sequence including the sequence of nucleotides 228-2764 of SEQ ID NO: 15.

In some embodiments, the nucleic acid vector of the invention includes a GFAP promoter of SEQ ID NO: 8 operably linked to a polynucleotide sequence encoding human Atoh1 having the amino acid sequence of SEQ ID NO: 10 (e.g., the polynucleotide of SEQ ID NO: 11). In some more specific embodiments, the nucleic acid vector of the invention includes, in 5′ to 3′ order, a first inverted terminal repeat; a GFAP promoter of SEQ ID NO: 8; a polynucleotide sequence encoding human Atoh1 having the amino acid sequence of SEQ ID NO: 10 operably linked to the GFAP promoter; a polyadenylation sequence; and a second inverted terminal repeat. In further, more specific embodiments, the nucleic acid vector includes, in 5′ to 3′ order, a first inverted terminal repeat; a GFAP promoter of SEQ ID NO: 8; a polynucleotide sequence encoding human Atoh1 having the amino acid sequence of SEQ ID NO: 10 operably linked to the GFAP promoter; a Woodchuck posttranscriptional regulatory element (WPRE); a polyadenylation sequence; and a second inverted terminal repeat. In some specific embodiments, the polynucleotide sequence encoding human Atoh1 is nucleotides 925-1986 of SEQ ID NO: 15. In even more specific embodiments, the nucleic acid vector includes nucleotides 228-2764 of SEQ ID NO: 15, flanked by inverted terminal repeats. In even more specific embodiments, the nucleic acid vector includes nucleotides 228-2764 of SEQ ID NO: 15, flanked by inverted terminal repeats, in which the 5′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 1-130 of SEQ ID NO: 15; and in which the 3′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 2852-2981 of SEQ ID NO: 15.

In some embodiments, the nucleic acid vector is a viral vector, plasmid, cosmid, or artificial chromosome. In some embodiments, the nucleic acid vector is a viral vector selected from the group including an AAV vector, an adenoviral vector, and a lentiviral vector. In some embodiments, the viral vector is an AAV vector. In some embodiments, the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S capsid. In some embodiments, the AAV vector has an AAV1 capsid. In some embodiments, the AAV vector has an AAV9 capsid. In some embodiments, the AAV vector has a 7m8 capsid. In some embodiments, the AAV vector has a PHP.S capsid. In some embodiments, the AAV vector has an Anc80 capsid. In some embodiments, the AAV vector has an Anc80L65 capsid. In some embodiments, the AAV vector has an AAV2 capsid. In some embodiments, the AAV vector has an AAV2quad(Y-F) capsid. In some embodiments, the AAV vector has a PHP.eB capsid. In some embodiments, the AAV vector has an AAV3 capsid. In some embodiments, the AAV vector has an AAV4 capsid. In some embodiments, the AAV vector has an AAV5 capsid. In some embodiments, the AAV vector has an AAV6 capsid. In some embodiments, the AAV vector has an AAV7 capsid. In some embodiments, the AAV vector has an AAV8 capsid. In some embodiments, the AAV vector has a PHP.B capsid.

It should be understood by those of ordinary skill in the art that the creation of a viral vector of the invention typically requires the use of a plasmid of the invention together with additional plasmids that provide required elements for proper viral packaging and viability (e.g., for AAV, plasmids providing the appropriate AAV rep gene, cap gene and other genes (e.g., E2A and E4)). The combination of those plasmids in a producer cell line produces the viral vector. However, it will be understood by those of skill in the art, that for any given pair of inverted terminal repeat sequences in a transfer plasmid of the invention (e.g., SEQ ID NO: 14 or 15) that is used to create the viral vector, the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the viral vector of the invention comprises nucleotides 1-2981 of SEQ ID NO: 15.

In another aspect, the invention provides a composition including a nucleic acid vector of the invention. In some embodiments, composition further includes a pharmaceutically acceptable carrier, diluent, or excipient.

In another aspect, the invention provides a cell containing a nucleic acid vector of the invention. In some embodiments, the cell is a mammalian supporting cell. In some embodiments, the mammalian supporting cell is a human supporting cell. In some embodiments, the supporting cell is a vestibular supporting cell (VSC) or a cochlear supporting cell.

In another aspect, the invention provides a method of expressing Atoh1 in a mammalian supporting cell by contacting the supporting cell with a nucleic acid vector or composition of the invention.

In some embodiments, the mammalian cell is a human supporting cell. In some embodiments, the mammalian supporting cell is a VSC or a cochlear supporting cell.

In another aspect, the invention provides a method of inducing or increasing hair cell regeneration in a subject in need thereof by administering to the subject an effective amount of a nucleic acid vector or composition of the invention.

In another aspect, the invention provides a method of inducing or increasing hair cell maturation in a subject in need thereof by administering to the subject an effective amount of a nucleic acid vector or composition of the invention. In some embodiments, the hair cell is a regenerated hair cell.

In another aspect, the invention provides a method of treating a subject having or at risk of developing vestibular dysfunction by administering to the subject an effective amount of a nucleic acid vector or composition of the invention.

In another aspect, the invention provides a method of treating a subject having or at risk of developing bilateral vestibulopathy (bilateral vestibular hypofunction) by administering to the subject an effective amount of a nucleic acid vector or composition of the invention.

In another aspect, the invention provides a method of treating a subject having or at risk of developing oscillopsia by administering to the subject an effective amount of a nucleic acid vector or composition of the invention.

In another aspect, the invention provides a method of treating a subject having or at risk of developing a balance disorder by administering to the subject an effective amount of a nucleic acid vector or composition of the invention.

In another aspect, the invention provides a method of treating a subject having or at risk of developing hearing loss (e.g., sensorineural hearing loss) by administering to the subject an effective amount of a nucleic acid vector or composition of the invention.

In another aspect, the invention provides a method of treating a subject having or at risk of developing tinnitus by administering to the subject an effective amount of a nucleic acid vector or composition of the invention.

In some embodiments of any of the foregoing aspects, the subject is a human.

In another aspect, the invention provides a human cell containing nucleic acid vector encoding a high expression promoter operably linked to a polynucleotide encoding Atoh1. In some embodiments, the cell is a supporting cell. In some embodiments, the supporting cell is a VSC or a cochlear supporting cell.

In another aspect, the invention provides a method of expressing Atoh1 in a human supporting cell by contacting the supporting cell with nucleic acid vector encoding a high expression promoter operably linked to a polynucleotide encoding Atoh1. In some embodiments, the supporting cell is a VSC or a cochlear supporting cell.

In another aspect, the invention provides a method of inducing or increasing hair cell regeneration in a human subject in need thereof by administering to the subject an effective amount of a nucleic acid vector encoding a high expression promoter operably linked to a polynucleotide encoding Atoh1.

In another aspect, the invention provides a method of inducing or increasing hair cell maturation in a human subject in need thereof by administering to the subject an effective amount of a nucleic acid vector encoding a high expression promoter operably linked to a polynucleotide encoding Atoh1.

In some embodiments of any of the foregoing aspects, the hair cell is a vestibular hair cell. In some embodiments, the vestibular hair cell is a Type II vestibular hair cell.

In some embodiments of any of the foregoing aspects, the hair cell is a cochlear hair cell. In some embodiments, the cochlear hair cell is an inner hair cell. In some embodiments, the cochlear hair cell is an outer hair cell.

In some embodiments of any of the foregoing aspects, the subject has or is at risk of developing vestibular dysfunction.

In some embodiments of any of the foregoing aspects, the subject has or is at risk of developing hearing loss (e.g., sensorineural hearing loss).

In another aspect, the invention provides a method of treating a human subject having or at risk of developing vestibular dysfunction by administering to the subject an effective amount of a nucleic acid vector encoding a high expression promoter operably linked to a polynucleotide encoding Atoh1.

In some embodiments of any of the foregoing aspects, the vestibular dysfunction is vertigo, dizziness, imbalance, bilateral vestibulopathy (bilateral vestibular hypofunction), oscillopsia, or a balance disorder.

In some embodiments, of any of the foregoing aspects, the vestibular dysfunction is age-related vestibular dysfunction, head trauma-related vestibular dysfunction, disease or infection-related vestibular dysfunction, or ototoxic drug-induced vestibular dysfunction.

In some embodiments of any of the foregoing aspects, the vestibular dysfunction is associated with a genetic mutation.

In some embodiments of any of the foregoing aspects, the vestibular dysfunction is idiopathic vestibular dysfunction.

In another aspect, the invention provides a method of treating a human subject having or at risk of developing bilateral vestibulopathy by administering to the subject an effective amount of a nucleic acid vector encoding a high expression promoter operably linked to a polynucleotide encoding Atoh1.

In some embodiments of any of the foregoing aspects, the bilateral vestibulopathy is ototoxic drug-induced bilateral vestibulopathy.

In another aspect, the invention provides a method of treating a human subject having or at risk of developing oscillopsia by administering to the subject an effective amount of a nucleic acid vector encoding a high expression promoter operably linked to a polynucleotide encoding Atoh1.

In some embodiments of any of the foregoing aspects, the oscillopsia is ototoxic drug-induced oscillopsia.

In another aspect, the invention provides a method of treating a human subject having or at risk of developing a balance disorder by administering to the subject an effective amount of a nucleic acid vector encoding a high expression promoter operably linked to a polynucleotide encoding Atoh1.

In another aspect, the invention provides a method of treating a human subject having or at risk of developing hearing loss by administering to the subject an effective amount of a nucleic acid vector encoding a high expression promoter operably linked to a polynucleotide encoding Atoh1.

In some embodiments of any of the foregoing aspects, the hearing loss is genetic hearing loss.

In some embodiments, the genetic hearing loss is autosomal dominant hearing loss, autosomal recessive hearing loss, or X-linked hearing loss.

In some embodiments of any of the foregoing aspects, the hearing loss is acquired hearing loss.

In some embodiments, the acquired hearing loss is noise-induced hearing loss, age-related hearing loss, disease or infection-related hearing loss, head trauma-related hearing loss, or ototoxic drug-induced hearing loss.

In another aspect, the invention provides a method of treating a human subject having or at risk of developing tinnitus by administering to the subject an effective amount of a nucleic acid vector encoding a high expression promoter operably linked to a polynucleotide encoding Atoh1.

In some embodiments of any of the foregoing aspects, the ototoxic drug is an aminoglycoside, an antineoplastic drug, ethacrynic acid, furosemide, a salicylate, or quinine.

In some embodiments of any of the foregoing aspects, the method further includes evaluating the vestibular function of the subject prior to administering the nucleic acid vector or composition.

In some embodiments of any of the foregoing aspects, the method further includes evaluating the vestibular function of the subject after administering the nucleic acid vector.

In some embodiments of any of the foregoing aspects, the method further includes evaluating the hearing of the subject prior to administering the nucleic acid vector.

In some embodiments of any of the foregoing aspects, the method further includes evaluating the hearing of the subject after administering the nucleic acid vector.

In some embodiments of any of the foregoing aspects, the nucleic acid vector is locally administered. In some embodiments, the nucleic acid vector is administered to the inner ear. In some embodiments, the nucleic acid vector is administered to the middle ear. In some embodiments, the nucleic acid vector is administered to a semicircular canal. In some embodiments, the nucleic acid vector is administered transtympanically or intratympanically. In some embodiments, the nucleic acid vector is administered into the perilymph. In some embodiments, the nucleic acid vector is administered into the endolymph. In some embodiments, the nucleic acid vector is administered to or through the oval window. In some embodiments, the nucleic acid vector is administered to or through the round window.

In some embodiments of any the foregoing aspects, the nucleic acid vector encoding a high expression promoter operably linked to a polynucleotide encoding Atoh1 is a nucleic acid vector containing a high expression, supporting cell-specific promoter described herein.

In some embodiments of any the foregoing aspects, the nucleic acid vector or composition is administered in an amount sufficient to prevent or reduce vestibular dysfunction, delay the development of vestibular dysfunction, slow the progression of vestibular dysfunction, improve vestibular function, prevent or reduce hearing loss, prevent or reduce tinnitus, delay the development of hearing loss, slow the progression of hearing loss, improve hearing, increase vestibular and/or cochlear hair cell numbers, increase vestibular and/or cochlear hair cell maturation, or increase vestibular and/or cochlear hair cell regeneration.

In another aspect, the invention provides a kit containing a nucleic acid vector or composition of the invention.

### Definitions

As used herein, the term “about” refers to a value that is within 10% above or below the value being described.

As used herein, “administration” refers to providing or giving a subject a therapeutic agent (e.g., a nucleic acid vector containing a high expression promoter, such as a high expression, supporting cell-specific promoter (e.g., a glial fibrillary acidic protein (GFAP) promoter having the sequence of formula A-B-C, in which all or part of B is optionally absent, such as a GFAP promoter having the sequence of SEQ ID NO: 8, operably linked to a polynucleotide encoding atonal BHLH transcription factor 1 (Atoh1)), by any effective route. Exemplary routes of administration are described herein below.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating hearing loss or vestibular dysfunction, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art. Also, as used herein, a “therapeutically effective amount” of a composition, vector construct, or viral vector of the present disclosure is an amount that results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human vestibular or cochlear supporting cell). Exogenous materials include those that are provided from an external source to an organism or to cultured matter extracted there from.

As used herein, the term “heterologous” refers to a combination of elements that is not naturally occurring. For example, a heterologous transgene refers to a transgene that is not naturally expressed by the promoter to which it is operably linked.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., cochlear or vestibular hair cell regeneration) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the middle or inner ear, and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules containing nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene.

As used herein, the term “high expression promoter” refers to a promoter that drives transgene expression in supporting cells transformed with a vector encoding such promoter operably linked to the transgene at a level that is at least 0.25 log fold change higher than expression of the transgene in supporting cells transformed with the same amount (e.g., titer) of an equivalent vector encoding the long GFAP promoter operably linked to the transgene. In some embodiments, a high expression promoter can express the transgene in supporting cells at a level that is at least 0.25, 0.5, 0.75, 1.0, 1.25, 1.5, 1.75, or 2.0 log fold change higher than the long GFAP promoter.

As used herein, the term “supporting cell-specific promoter” refers to a promoter that leads to GFP immunolabeling above background in at least 50% of supporting cells and in less than 20% of hair cells in the method for identifying a supporting cell-specific promoter described herein.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the terms “subject” and “patient” refer to an animal (e.g., a mammal, such as a human). A subject to be treated according to the methods described herein may be one who has been diagnosed with vestibular dysfunction (e.g., dizziness, vertigo, imbalance, bilateral vestibulopathy (bilateral vestibular hypofunction), or oscillopsia) and/or hearing loss (e.g., sensorineural hearing loss) or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Lorence, Recombinant Gene Expression: Reviews and Protocols (Humana Press, New York, N.Y., 2012).

As used herein, the term “transfection” refers to any of a wide variety of techniques commonly used for the introduction of exogenous DNA into a prokaryotic or eukaryotic host cell, e.g., electroporation, lipofection, calcium phosphate precipitation, DEAE-dextran transfection, Nucleofection, squeeze-poration, sonoporation, optical transfection, magnetofection, impalefection and the like.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, M A, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “vestibular hair cell” refers to a type of specialized cell in the inner ear that is involved in sensing movement and contributes to the sense of balance and spatial orientation. There are two types of vestibular hair cells: Type I and Type II hair cells. Type I hair cells have calyx nerve endings, fast voltage responses, and encode dynamic movements. Type II hair cells have bouton nerve endings, slower voltage responses, and encode slow or static movements. Vestibular hair cells are located in the semicircular canal end organs and otolith organs of the inner ear. Damage to vestibular hair cells and genetic mutations that disrupt vestibular hair cell function are implicated in vestibular dysfunction such as vertigo and imbalance disorders.

As used herein, the term “supporting cell” refers specialized epithelial cells in the cochlea and vestibular system of the inner ear that reside between hair cells. Supporting cells maintain the structural integrity of the sensory organs during sound stimulation and head movements and help to maintain an environment in the epithelium that allows hair cells to function. Supporting cells are also involved in cochlear and vestibular hair cell development, survival, death, and phagocytosis.

As used herein, the term “wild-type” refers to a genotype with the highest frequency for a particular gene in a given organism.

### BRIEF DESCRIPTION OF THE DRAWINGS

FIGS. 1A-1C are a series of confocal images and graphs showing that AAV-Atoh1 regenerates utricular hair cells in a dose-dependent manner. Utricles were dissected from male C57Bl/6J mice (6-8-week-old) and cultured in 100 μL of base medium. Gentamicin (0.5 mg/mL) was added to the medium for 24 hours to kill hair cells, after which the gentamicin was washed out and replaced with 1 mL fresh medium for 3 days. AAV1-CMV-Atoh1-2A-H2BGFP was then added to the culture medium at 4×10⁷ genome copies (gc)/mL, 4×10⁸ gc/mL, 4×10⁹ gc/mL, 4×10¹⁰ gc/mL, or 4×10¹¹ gc/mL. After 3 days of incubation, virus was washed out and utricles were cultured for an additional 5 days in 2 mL of fresh medium, and then fixed and immunostained with antibodies to Pou4f3 and Sall2. A dose dependent increase in hair cell regeneration was observed in utricular explants treated with increasing doses of virus (FIG. 1A). Anti-Pou4f3 labeling was used to visualize hair cells. Insets show GFP expression throughout the utricle, which also increased with viral dose. The number of hair cells (Pou4f3+ nuclei) and supporting cells (Sall2+ nuclei) per utricle were graphed as a function of viral dose (FIGS. 1B-1 C). The increase in hair cells was fit by a one phase exponential association (FIG. 11B). Supporting cell numbers decreased with viral dose, indicating that hair cell regeneration was occurring via direct conversion of supporting cells into hair cells without an intervening mitosis (FIG. 1C).

FIGS. 2A-2C are a series of confocal images and graphs showing that the level of Atoh1 overexpression correlates with the efficiency of supporting-cell-to-hair-cell conversion. Utricles were dissected from male C57Bl/6J mice (6-8-week-old) and cultured in 100 μL of base medium. Gentamicin (0.5 mg/mL) was added to the medium for 24 hours to kill hair cells, after which the gentamicin was washed out and replaced with 250 μL fresh medium containing one of the following AAV vectors at a dose of 1 E12 genome copies (gc): AAV8-CMV-Atoh1-2A-H2BGFP (very high expression), AAV8-GFAP(SEQ ID NO: 8; “short GFAP” promoter)-Atoh1-2A-H2BGFP (high expression), AAV8-RLBP1-Atoh1-2A-H2BGFP (low expression). After 1 day of incubation, virus was washed out and utricles were cultured for an additional 7 days in 2 mL of fresh medium, and then fixed and immunostained with antibodies to Pou4f3. Alternatively, some utricles were dissociated and single cells were captured and prepared for single-cell RNA sequencing (scRNA-Seq). Anti-Pou4f3 labeling and native GFP signal were imaged in the utricles treated with AAV vectors encoding Atoh1 driven by promoters with high (CMV), medium (GFAP), or low (RLBP1) levels of activity in supporting cells. At equal viral doses, promoters that induced higher levels of Atoh1 expression stimulated higher levels of hair cell regeneration (FIG. 2A). The difference in promoter activity was observed via the H2BGFP signal (FIG. 2A middle panel, microscope acquisition setting equal across all conditions). Adjusting the microscope acquisition settings to match the GFP intensity level (FIG. 2A right panel) revealed that viral transduction was comparable and widespread throughout the sensory epithelium for each virus, despite the differences in expression level. FIG. 2B shows a quantification of hair cell counts (Pou4f3+ nuclei) from each condition. Single-cell RNA-Seq data were graphed using violin plots to show the levels of Atoh1 transgene expression in supporting cells from each condition (FIG. 2C). The expression analysis confirmed the gradient in promoter activity across the three viruses.

FIG. 3 is a graph showing that the short GFAP promoter induced higher levels of transgene expression than a GFAP promoter having the sequence of SEQ ID NO: 9 (“long GFAP” promoter; positions −2163 to +47 relative to the transcriptional start site of the human GFAP gene) in U87 cells. U87 human glioblastoma cells were seeded at a density of 10,000 cells/well in a 96-well plate. One day after seeding, 100 ng of plasmid encoding either short GFAP-H2BGFP (plasmid P332) or long GFAP-H2BGFP (plasmid P378) was transfected into the cells. Non-transfected cells (NT) were used as a control. Two days after transfection, the cells were dissociated and the percentage of GFP+ cells for each condition was determined with flow cytometry on a Sony SH800 FACS machine. A higher percentage of GFP+ cells were detected with the short GFAP promoter compared to the long GFAP promoter (FIG. 3). Cells were transfected with equal amounts of plasmid, indicating that the increased detection rate was driven by higher levels of transgene expression, and, therefore, more cells with GFP levels surpassing the detection limit of the FACS machine.

FIGS. 4A-4B are a series of confocal images showing that short GFAP promoter induced higher levels of transgene expression than long GFAP promoter in utricle explants. Utricles were dissected from male C57Bl/6J mice (11-week-old) and placed into 250 μL of culture medium containing DMEM/F12, 5% FBS, 2.5 μg/mL ciprofloxacin, and 2.5E11 gc of AAV8-short GFAP-H2BGFP or AAV8-long GFAP-H2BGFP at 37° C. and 5% CO₂. After 1 day of incubation, virus was washed out and utricles were cultured for an additional 6 days in 2 mL of fresh medium, and then fixed and imaged. The intensity of H2BGFP in supporting cells was higher in the utricles (U) and cristae (C) treated with AAV vectors encoding the short GFAP promoter (FIG. 4A) compared to the long GFAP promoter (FIG. 4B). Since the AAV vectors were transduced at equal doses, these data indicate that the short GFAP promoter drives higher levels of expression in supporting cells compared to the long GFAP promoter.

FIG. 5 is a series of images showing that the short GFAP promoter is active in mouse vestibular supporting cells in vivo. AAV8-short GFAP-H2BGFP was injected into the left posterior canal of C56B1/6J mice (6-8-week-old) at a dose of 1.51 E10 gc/ear (1 μL total volume injected). Fourteen days later, mice were sacrificed and fixed with formalin via cardiac perfusion. Temporal bones were removed, decalcified in EDTA, embedded in paraffin, and sectioned on a microtome. Slides were stained with chromogenic antibodies to GFP and haemotoxylin (H&E). Sections were imaged with a Leica Aperio digital slide scanner. Intense nuclear GFP labeling was detected in all supporting cells from both the utricle (left) and the crista (right), but not in hair cells.

FIGS. 6A-6B are a series of images and graphs showing that AAV8-short GFAP-Atoh1 robustly regenerates vestibular hair cells in vivo. A single I.P. injection of 5 g/kg 3,3′-iminodipropanenitrile (IDPN) was delivered to 8-9-week-old CD-1 mice (n=6). Fifteen to seventeen days later, 1 μL of AAV8-short GFAP-Atoh1-2A-H2BGFP at a dose of 7.2E9 vg was delivered to the posterior semicircular canal (left ear only). Mice were allowed to survive for 13-14 days after virus delivery and then sacrificed and fixed with formalin via cardiac perfusion. Vestibular organs were microdissected and processed for immunohistochemistry. Organs were dissected from the ear of a naïve mouse not treated with IDPN or AAV (left), the contralateral ear of an IDPN-damaged mouse not treated with virus (middle), or the AAV-treated ear from the same IDPN-damaged mouse. Confocal images of utricles (FIG. 6A, top row) and cristae (FIG. 6A, bottom row) immunolabeled with antibodies to Pou4f3 are shown. IDPN caused a substantial decrease in hair cell numbers; however, treating with AAV8-short GFAP-ATOH1 after IDPN damage robustly regenerated hair cells in both the utricle and cristae (FIG. 6A). Hair cell numbers in utricles and cristae treated with AAV8-short GFAP-Atoh1 after IDPN damage were quantified (based on Pou4f3 labeling) and compared to hair cell numbers in contralateral ears that were not treated with AAV (FIG. 6B); p<0.001, paired t-test.

FIG. 7 is a series of images showing that stereocilia bundle density is increased in regenerated utricles. A single I.P. injection of 5 g/kg IDPN was delivered to 8-week-old CD-1 mice (n=12). Fourteen to twenty-two days later, 1 μL of AAV8-short GFAP-ATOH1-2A-H2BGFP at a dose of 2.5E10 vg was delivered to the posterior semicircular canal (left ear only). Mice were allowed to survive for 30 days after virus delivery and then sacrificed and fixed with formalin via cardiac perfusion. Vestibular organs were microdissected and processed for immunohistochemistry. Confocal images were collected to visualize fluorescent phalloidin labeling of F-actin in a utricle from an IDPN-damaged mouse that received AAV8-short GFAP-ATOH1 in its left ear (FIG. 7, left). A higher density of stereocilia bundles was observed in this utricle compared to the utricle from the contralateral ear (FIG. 7, right) that did not receive virus. The inset image in the left panel shows GFP expression, confirming successful delivery of the virus.

FIG. 8 is a series of images showing nerve fiber and synapse density are increased in regenerated utricles. A single I.P. injection of 5 g/kg IDPN was delivered to 8-week-old CD-1 mice (n=12). Fourteen to twenty-two days later, 1 μL of AAV8-short GFAP-ATOH1-2A-H2BGFP at a dose of 2.5E10 vg was delivered to the posterior semicircular canal (left ear only). Mice were allowed to survive for 14 days after virus delivery and then sacrificed and fixed with formalin via cardiac perfusion. Vestibular organs were microdissected and processed for immunohistochemistry. Confocal images show immunostaining for Myo7a (hair cells), Nefh (nerve fibers), and Ctbp2 (ribbon synapses) in a utricle from an IDPN-damaged mouse that received AAV8-short GFAP-ATOH1 in its left ear (FIG. 8, left). A higher density of nerve fibers and ribbon synapses was observed in this utricle compared to the utricle from the contralateral ear (FIG. 8, right) that did not receive virus.

FIGS. 9A-9D are a series of graphs showing that silencing Atoh1 transgene expression in new hair cells via a supporting-cell-specific promoter drives further maturation. Utricles were dissected from male C57Bl/6J mice (6-8-week-old) and cultured in 100 μl of base medium. Gentamicin (0.5 mg/mL) was added to the medium for 24 hours to kill hair cells, after which the gentamicin was washed out and replaced with 250 μL fresh medium containing one of the following AAVs at a dose of 1 E12 go: AAV8-CMV-Atoh1-2A-H2BGFP (CMV promoter group), AAV8-short GFAP-Atoh1-2A-H2BGFP (supporting cell (SC)-specific promoter group), or AAV8-RLBP1-Atoh1-2A-H2BGFP (SC-specific promoter group). After one day of incubation, virus was washed out and utricles were cultured for an additional 3, 8, or 16 days in 2 mL of fresh medium. At the end of the culture period, utricles were dissociated and single cells were captured and prepared for single-cell RNA-Seq. Prediction scores were generated in Seurat by comparing to databases of utricle hair cell single-cell RNA-Seq profiles that were generated from embryonic day 18 (E18), postnatal day 12 (P12), and adult mice. Violin plots were generated to show Atoh1 transgene expression and maturity prediction scores for regenerated hair cells in adult utricle explants. The Atoh1 transgene was expressed at low or undetectable levels in regenerated hair cells in the SC-specific promoter group, whereas it was expressed at high levels in almost all hair cells from the CMV group (FIG. 9A). These results demonstrate that the Atoh1-transgene naturally downregulates in regenerated hair cells when it is driven by a SC-specific promoter. In addition, more of the single-cell RNA-Seq profiles from the SC-specific promoter group correlated strongly with P12 (FIG. 9C) and adult hair cells (FIG. 9D) than those from the CMV group. Conversely, more of the single-cell RNA-Seq profiles from the CMV group correlated strongly with E18 hair cells (FIG. 9B) than those from the SC-specific promoter group. Thus, natural silencing of the Atoh1 transgene with a SC-specific promoter induced maturation of regenerated hair cells.

FIGS. 10A-10D are a series of graphs showing that “low” and “high” levels of Atoh1 expression in supporting cells generate two distinct populations of hair cells. Utricles were dissected from male C57Bl/6J mice (6-8-week-old) and cultured in 100 μL of base medium. Gentamicin (0.5 mg/mL) was added to the medium for 24 hours to kill hair cells, after which the gentamicin was washed out and replaced with 250 μL fresh medium containing one of the following AAVs at a dose of 1 E12 gc: AAV8-CMV-Atoh1-2A-H2BGFP, AAV8-short GFAP-Atoh1-2A-H2BGFP, or AAV8-RLBP1-Atoh1-2A-H2BGFP. After one day of incubation, virus was washed out and utricles were cultured for an additional 3, 8, or 16 days in 2 mL of fresh medium. At the end of the culture period, utricles were dissociated and single cells were captured and prepared for single-cell RNA-Seq. Prediction scores were generated in Seurat by comparing to databases of utricle hair cell single-cell RNA-Seq profiles that were generated from E18 and P12 mice. FIG. 10A shows a UMAP plot of single-cell RNA-Seq expression profiles generated from supporting cells and regenerated hair cells. The supporting cells separated into two distinct clusters (labeled as Supporting Cells 1 and Supporting Cells 2) from which two clusters of regenerated hair cells appeared to originate (FIG. 10A, left). The supporting cells in cluster 1 were made up almost entirely of cells from samples treated with the short GFAP and CMV viruses, whereas almost all the supporting cells from the samples treated with RLBP1 virus fell in cluster 2 (FIG. 10A, right). Violin plots were produced to show Atoh1 transgene expression in the two supporting cell groups and demonstrated that cluster 1 had substantially higher levels of transgene expression compared to cluster 2 (FIG. 10B). Since both cluster 1 and 2 were made up of large numbers of cells from both the CMV and short GFAP virus conditions, the separation of the clusters was driven more by the difference in Atoh1 expression level as opposed to treatment type. RLBP1 is the weakest of the three promoters and almost no cells from this treatment group reached high enough levels of Atoh1 expression to fall into cluster 1. Violin plots were also produced to show maturity prediction scores for regenerated hair cells from the two hair cell clusters (FIGS. 10C-10D). More of the single-cell RNA-Seq profiles from hair cell cluster 1 (hair cells generated from supporting cells with high levels of Atoh1 expression) correlated strongly with P12 hair cells than those from cluster 2 (FIG. 10D). Conversely, more of the single-cell RNA-Seq profiles from cluster 2 (hair cells generated from supporting cells with low levels of Atoh1 expression) correlated strongly with E18 hair cells than those from cluster 1 (FIG. 10C). Thus, higher levels of Atoh1 expression in supporting cells appeared to generate more mature hair cells than lower levels of Atoh1 expression. Weak promoters like RLBP1 were not able to drive sufficiently high levels of Atoh1 to generate many mature hair cells at the AAV doses used in this experiment.

FIG. 11 is a schematic showing an alignment of the long GFAP promoter to the GFAP promoter elements described herein. Nucleotide positions are labeled relative to the transcription start site of the human GFAP gene.

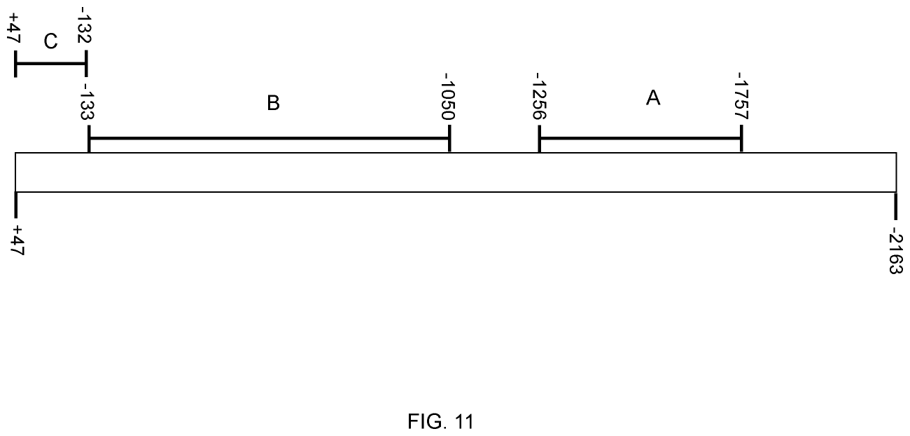

FIG. 12 is a graph showing that the number of utricular hair cells (as determined with Pou4f3 immunolabeling) in ears treated with AAV8-short GFAP-hAtoh1 (human ATOH1 transgene without a GFP tag) after IDPN damage was significantly greater than the number of utricular hair cells in contralateral ears that were not treated with AAV; p<0.05, paired t-test.

FIG. 13 is a map of the plasmid P377 having the sequence set forth in SEQ ID NO:14, which is useful to synthesize AAV8-short GFAP-hAtoh1-2A-H2BGFP.

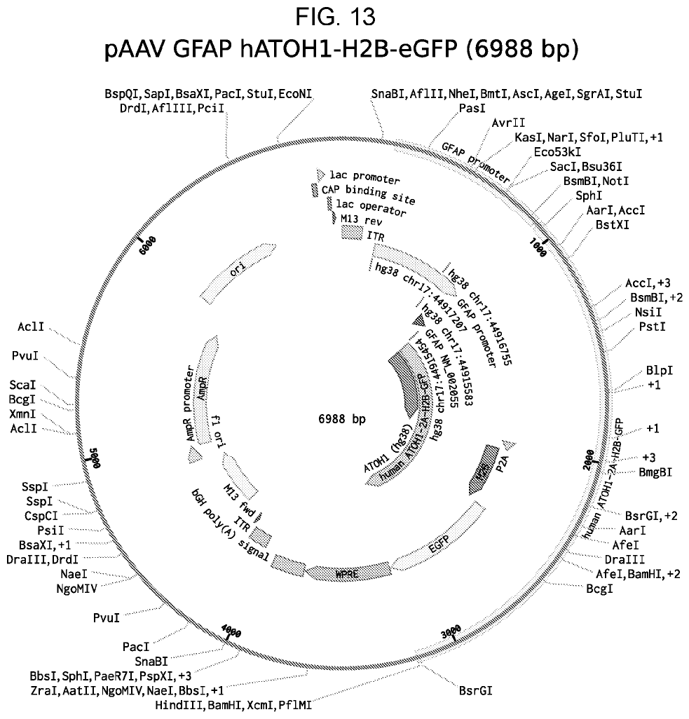

FIG. 14 is a map of the plasmid P712 having the sequence set forth in SEQ ID NO:15, which is useful to synthesize AAV8-short GFAP-hAtoh1.

FIG. 15 is a map of plasmid P332.

FIG. 16 is a map of plasmid P378.

FIG. 17 is a map of plasmid P319, which is useful to synthesize AAV8-short GFAP-mouse Atoh1-2A-H2BGFP

### DETAILED DESCRIPTION

Described herein are compositions and methods for inducing hair cell regeneration (e.g., cochlear and/or vestibular hair cell regeneration). The invention features nucleic acid vectors (e.g., viral vectors, such as adeno-associated virus (AAV) vectors) containing a high expression promoter, such as a high expression, supporting cell-specific promoter (e.g., a GFAP promoter having the sequence of SEQ ID NO: 8), operably linked to a polynucleotide encoding Atoh1. The nucleic acid vectors described herein can be used to express Atoh1 in supporting cells (e.g., cochlear and/or vestibular supporting cells) to promote hair cell regeneration (e.g., cochlear and/or vestibular hair cell regeneration). Therefore, the compositions described herein can be administered to a subject (a mammalian subject, for example, a human) to treat disorders caused by loss of or damage to cochlear hair cells, such as hearing loss (e.g., sensorineural hearing loss), or disorders caused by loss of or damage to vestibular hair cells, such as dizziness, vertigo, imbalance, bilateral vestibulopathy (bilateral vestibular hypofunction), oscillopsia, and balance disorders.

### Hair Cell Regeneration

Hair cells are sensory cells of the auditory and vestibular systems that reside in the inner ear. Cochlear hair cells are the sensory cells of the auditory system and are made up of two main cell types: inner hair cells, which are responsible for sensing sound, and outer hair cells, which are thought to amplify low-level sound. Vestibular hair cells are located in the semicircular canal end organs and otolith organs of the inner ear and are involved in the sensation of movement that contributes to the sense of balance and spatial orientation. Hair cells are named for the stereocilia that protrude from the apical surface of the cell, forming a hair cell bundle. Deflection of the stereocilia (e.g., by sound waves in cochlear hair cells, or by rotation or linear acceleration in vestibular hair cells) leads to the opening of mechanically gated ion channels, which allows hair cells to release neurotransmitters to activate nerves, thereby converting mechanical sound or motion signals into electrical signals that can be transmitted to the brain. Cochlear hair cells are essential for normal hearing, and damage to or loss of cochlear hair cells and genetic mutations that disrupt cochlear hair cell function are implicated in hearing loss and deafness. Damage to or loss of vestibular hair cells and genetic mutations that disrupt vestibular hair cell function are implicated in vestibular dysfunction, such as dizziness, vertigo, balance loss, bilateral vestibulopathy (bilateral vestibular hypofunction), oscillopsia, and balance disorders.

There are various factors that can contribute to hair cell loss or damage. One common factor is aging, which can contribute to both cochlear and vestibular hair cell loss. Histopathology from temporal bone specimens reveals that humans lose vestibular hair cells with age, leading to an age-related decline in various measures of vestibular function, such as the vestibulo-ocular reflex (VOR). A decline in vestibular performance, such as VOR, is well-correlated with fall risk in the elderly. Hair cells may also be damaged by insults such as ototoxins (e.g., aminoglycoside antibiotics), infections (e.g., viral infections), trauma (e.g., head trauma or exposure to loud noise), and autoimmune responses (e.g., in subjects having an autoimmune disease or condition). Supporting cells and other structures in the inner ear can remain intact despite hair cell loss, but hearing and/or vestibular function are greatly diminished.

There is some spontaneous regeneration of hair cells that occurs in the vestibular system of mammals. However, less than 20% of vestibular hair cells spontaneously regenerate, which is insufficient for functional recovery. In addition, little to no spontaneous hair cell regeneration is thought to occur in the mammalian cochlea. Thus, there is a need for therapeutics that can increase vestibular and/or cochlear hair cell regeneration to a level that restores or improves vestibular function and/or hearing.

### Atoh1

Atoh1 is a basic helix-loop-helix transcription factor that has been implicated in the development of neuronal and epithelial cell types. In mice, knocking out Atoh1 prevents the development of hair cells, suggesting that Atoh1 is necessary for hair cell development. Atoh1 overexpression has also been found to lead to an increase in hair cell numbers, suggesting that Atoh1 is sufficient to promote the development of hair cells. Accordingly, various approaches to increase Atoh1 have been evaluated as potential therapeutics for treating hearing loss or vestibular dysfunction. As substantial hair cell regeneration is necessary to promote functional recovery, there is a need for new therapeutic approaches that induce a strong regenerative response and the formation of mature hair cells.

The present invention is based, in part, on the discovery that a gene therapy approach using a promoter that induced high expression of Atoh1 in supporting cells led to the regeneration of substantially more hair cells than a gene therapy approach using a promoter that induced low Atoh1 expression. In addition, the use of a promoter that induced high initial expression of Atoh1 in supporting cells was found to lead to the formation of more mature hair cells. Furthermore, a gene therapy approach using a promoter that induced high, supporting cell-specific expression of Atoh1 led to both robust regeneration and improved maturation of regenerated hair cells.

The compositions described herein include a nucleic acid vector containing a high expression promoter operably linked to a polynucleotide encoding Atoh1. In some embodiments, the high expression promoter is also a supporting cell-specific promoter. These compositions can be used to induce Atoh1 expression in supporting cells (e.g., cochlear and/or vestibular supporting cells) and to promote hair cell (e.g., cochlear and/or vestibular hair cell) regeneration and maturation. Hair cells produced using the compositions described herein generate new stereocilia bundles and form synapses. Accordingly, the compositions and methods described herein can be used to treat a subject having or at risk of developing hearing loss (e.g., sensorineural hearing loss) and/or vestibular dysfunction (e.g., dizziness, vertigo, imbalance, bilateral vestibulopathy (bilateral vestibular hypofunction), oscillopsia, or a balance disorder).

In some embodiments, the high expression, supporting cell-specific promoter included in a nucleic acid vector described herein is a GFAP promoter having the sequence of formula A-B-C, in which A has the sequence of SEQ ID NO: 1, B has the sequence of SEQ ID NO: 2, and C has the sequence of SEQ ID NO: 3, in which all or part of B is optionally absent. In some embodiments, all of B is present. In some embodiments, 1-100, 100-200, 200-300, 300-400, 400-500, 500-600, 600-700, 700-800, or 800-900 nucleotides of B are present (e.g., at least 1, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 915 nucleotides of B are present in the GFAP promoter having the sequence of formula A-B-C). In some embodiments, nucleotides 1-253 of B (corresponding to SEQ ID NO: 4) are present. In some embodiments, nucleotides 230-483 of B (corresponding to SEQ ID NO: 5) are present.

In some embodiments, nucleotides 459-711 of B (corresponding to SEQ ID NO: 6) are present. In some embodiments, nucleotides 687-917 of B (corresponding to SEQ ID NO: 7) are present. In other embodiments, all of B is absent. The GFAP promoter induces increased Atoh1 expression in supporting cells compared to Atoh1 expression induced in supporting cells by a GFAP promoter having the sequence of SEQ ID NO: 9. In some embodiments, the GFAP promoter increases Atoh1 expression in supporting cells by at least 0.25 log fold change (e.g., at least 0.25, 0.5, 0.75, 1.0, 1.25, 1.5, 1.75, or 2.0 log fold change) compared to Atoh1 expression induced in supporting cells by a GFAP promoter having the sequence of SEQ ID NO: 9. In some embodiments, the GFAP promoter has the sequence of SEQ ID NO: 8. The GFAP promoter sequences described above are provided in Table 2. Atoh1 sequences that can be included in or expressed by the compositions of the invention are provided in Table 3.

TABLE 2
GFAP promoter sequences

| SEQ | Description of | |
|---|---|---|
| ID | promoter | |
| NO: | sequence | Promoter sequence |
| 1 | Part A of the | AACATATCCTGGTGTGGAGTAGGGGACGCTGCTCTGACAGAGGCTC |
| | GFAP promoter | GGGGGCCTGAGCTGGCTCTGTGAGCTGGGGAGGAGGCAGACAGCC |
| | sequence | AGGCCTTGTCTGCAAGCAGACCTGGCAGCATTGGGCTGGCCGCCCC |
| | | CACAATGTTCGGGGTGGGCACAGTGCCTGCTTCCCGCCGCACCCCA |
| | | GCCCCCCTCAAATGCCTTCCGAGAAGCCCATTGAGCAGGGGGCTTG |
| | | CATTGCACCCCAGCCTGACAGCCTGGCATCTTGGGATAAAAGCAGCA |
| | | CAGCCCCCTAGGGGCTGCCCTTGCTGTGTGGCGCCACCGGCGGTG |
| | | GAGAACAAGGCTCTATTCAGCCTGTGCCCAGGAAAGGGGATCAGGG |
| | | GATGCCCAGGCATGGACAGTGGGTGGCAGGGGGGGAGAGGAGGGC |
| | | TGTCTGCTTCCCAGAAGTCCAAGGACACAAATGGGTGAGGGGA |
| 2 | Part B of the | AGGGGTGGCCAGGGAAACGGGGCGCTGCAGGAATAAAGACGAGCC |
| | GFAP promoter | AGCACAGCCAGCTCATGTGTAACGGCTTTGTGGAGCTGTCAAGGCCT |
| | sequence | GGTCTCTGGGAGAGAGGCACAGGGAGGCCAGACAAGGAAGGGGTG |
| | | ACCTGGAGGGACAGATCCAGGGGCTAAAGTCCTGATAAGGCAAGAG |
| | | AGTGCCGGCCCCCTCTTGCCCTATCAGGACCTCCACTGCCACATAGA |
| | | GGCCATGATTGACCCTTAGACAAAGGGCTGGTGTCCAATCCCAGCC |
| | | CCCAGCCCCAGAACTCCAGGGAATGAATGGGCAGAGAGCAGGAATG |
| | | TGGGACATCTGTGTTCAAGGGAAGGACTCCAGGAGTCTGCTGGGAA |
| | | TGAGGCCTAGTAGGAAATGAGGTGGCCCTTGAGGGTACAGAACAGG |
| | | TTCATTCTTCGCCAAATTCCCAGCACCTTGCAGGCACTTACAGCTGA |
| | | GTGAGATAATGCCTGGGTTATGAAATCAAAAAGTTGGAAAGCAGGTC |
| | | AGAGGTCATCTGGTACAGCCCTTCCTTCCCTTTTTTTTTTTTTTTTTTG |
| | | TGAGACAAGGTCTCTCTCTGTTGCCCAGGCTGGAGTGGCGCAAACA |
| | | CAGCTCACTGCAGCCTCAACCTACTGGGCTCAAGCAATCCTCCAGCC |
| | | TCAGCCTCCCAAAGTGCTGGGATTACAAGCATGAGCCACCCCACTCA |
| | | GCCCTTTCCTTCCTTTTTAATTGATGCATAATAATTGTAAGTATTCATC |
| | | ATGGTCCAACCAACCCTTTCTTGACCCACCTTCCTAGAGAGAGGGTC |
| | | CTCTTGCTTCAGCGGTCAGGGCCCCAGACCCATGGTCTGGCTCCAG |
| | | GTACCACCTGCCTCATGCAGGAGTTGGCGTGCCCAGGAAGCTCTGC |
| | | CTCTGGGCACAGTGACCTCAGTGGGGTGAGGG |
| 3 | Part C of the | GAGCTCTCCCCATAGCTGGGCTGCGGCCCAACCCCACCCCCTCAGG |
| | GFAP promoter | CTATGCCAGGGGGTGTTGCCAGGGGCACCCGGGCATCGCCAGTCTA |
| | sequence | GCCCACTCCTTCATAAAGCCCTCGCATCCCAGGAGCGAGCAGAGCC |
| | | AGAGCAGGTTGGAGAGGAGACGCATCACCTCCGCTGCTCGC |
| 4 | Nucleotides 1-254 | AGGGGTGGCCAGGGAAACGGGGCGCTGCAGGAATAAAGACGAGCC |
| | of Part B | AGCACAGCCAGCTCATGTGTAACGGCTTTGTGGAGCTGTCAAGGCCT |
| | | GGTCTCTGGGAGAGAGGCACAGGGAGGCCAGACAAGGAAGGGGTG |
| | | ACCTGGAGGGACAGATCCAGGGGCTAAAGTCCTGATAAGGCAAGAG |
| | | AGTGCCGGCCCCCTCTTGCCCTATCAGGACCTCCACTGCCACATAGA |
| | | GGCCATGATTGACCCTTAGACAAA |
| 5 | Nucleotides 230- | AGGCCATGATTGACCCTTAGACAAAGGGCTGGTGTCCAATCCCAGCC |
| | 483 of Part B | CCCAGCCCCAGAACTCCAGGGAATGAATGGGCAGAGAGCAGGAATG |
| | | TGGGACATCTGTGTTCAAGGGAAGGACTCCAGGAGTCTGCTGGGAA |
| | | TGAGGCCTAGTAGGAAATGAGGTGGCCCTTGAGGGTACAGAACAGG |
| | | TTCATTCTTCGCCAAATTCCCAGCACCTTGCAGGCACTTACAGCTGA |
| | | GTGAGATAATGCCTGGGTTATG |
| 6 | Nucleotides 459- | TGAGTGAGATAATGCCTGGGTTATGAAATCAAAAAGTTGGAAAGCAG |
| | 711 of Part B | GTCAGAGGTCATCTGGTACAGCCCTTCCTTCCCTTTTTTTTTTTTTTTT |
| | | TTGTGAGACAAGGTCTCTCTCTGTTGCCCAGGCTGGAGTGGCGCAAA |
| | | CACAGCTCACTGCAGCCTCAACCTACTGGGCTCAAGCAATCCTCCAG |
| | | CCTCAGCCTCCCAAAGTGCTGGGATTACAAGCATGAGCCACCCCACT |
| | | CAGCCCTTTCCTTCCT |
| 7 | Nucleotides 687- | CACCCCACTCAGCCCTTTCCTTCCTTTTTAATTGATGCATAATAATTGT |
| | 917 of Part B | AAGTATTCATCATGGTCCAACCAACCCTTTCTTGACCCACCTTCCTAG |
| | | AGAGAGGGTCCTCTTGCTTCAGCGGTCAGGGCCCCAGACCCATGGT |
| | | CTGGCTCCAGGTACCACCTGCCTCATGCAGGAGTTGGCGTGCCCAG |
| | | GAAGCTCTGCCTCTGGGCACAGTGACCTCAGTGGGGTGAGGG |
| 8 | GFAP promoter | AACATATCCTGGTGTGGAGTAGGGGACGCTGCTCTGACAGAGGCTC |
| | of formula A-C | GGGGGCCTGAGCTGGCTCTGTGAGCTGGGGAGGAGGCAGACAGCC |
| | (short GFAP | AGGCCTTGTCTGCAAGCAGACCTGGCAGCATTGGGCTGGCCGCCCC |
| | promoter) | CCAGGGCCTCCTCTTCATGCCCAGTGAATGACTCACCTTGGCACAGA |
| | | CACAATGTTCGGGGTGGGCACAGTGCCTGCTTCCCGCCGCACCCCA |
| | | GCCCCCCTCAAATGCCTTCCGAGAAGCCCATTGAGCAGGGGGCTTG |
| | | CATTGCACCCCAGCCTGACAGCCTGGCATCTTGGGATAAAAGCAGCA |
| | | CAGCCCCCTAGGGGCTGCCCTTGCTGTGTGGCGCCACCGGCGGTG |
| | | GAGAACAAGGCTCTATTCAGCCTGTGCCCAGGAAAGGGGATCAGGG |
| | | GATGCCCAGGCATGGACAGTGGGTGGCAGGGGGGGAGAGGAGGGC |
| | | TGTCTGCTTCCCAGAAGTCCAAGGACACAAATGGGTGAGGGGAGAG |
| | | CTCTCCCCATAGCTGGGCTGCGGCCCAACCCCACCCCCTCAGGCTA |
| | | TGCCAGGGGGTGTTGCCAGGGGCACCCGGGCATCGCCAGTCTAGC |
| | | CCACTCCTTCATAAAGCCCTCGCATCCCAGGAGCGAGCAGAGCCAG |
| | | AGCAGGTTGGAGAGGAGACGCATCACCTCCGCTGCTCGC |
| 9 | Long GFAP | GAGCTCCCACCTCCCTCTCTGTGCTGGGACTCACAGAGGGAGACCT |
| | promoter | CAGGAGGCAGTCTGTCCATCACATGTCCAAATGCAGAGCATACCCTG |
| | | GGCTGGGCGCAGTGGCGCACAACTGTAATTCCAGCACTTTGGGAGG |
| | | CTGATGTGGAAGGATCACTTGAGCCCAGAAGTTCTAGACCAGCCTGG |
| | | GCAACATGGCAAGACCCTATCTCTACAAAAAAAGTTAAAAAATCAGCC |
| | | ACGTGTGGTGACACACACCTGTAGTCCCAGCTATTCAGGAGGCTGA |
| | | GGTGAGGGGATCACTTAAGGCTGGGAGGTTGAGGCTGCAGTGAGTC |
| | | GTGGTTGCGCCACTGCACTCCAGCCTGGGCAACAGTGAGACCCTGT |
| | | CTCAAAAGACAAAAAAAAAAAAAAAAAAAAAAAGAACATATCCTGGTG |
| | | TGGAGTAGGGGACGCTGCTCTGACAGAGGCTCGGGGGCCTGAGCT |
| | | GGCTCTGTGAGCTGGGGAGGAGGCAGACAGCCAGGCCTTGTCTGCA |
| | | AGCAGACCTGGCAGCATTGGGCTGGCCGCCCCCCAGGGCCTCCTCT |
| | | TCATGCCCAGTGAATGACTCACCTTGGCACAGACACAATGTTCGGGG |
| | | TGGGCACAGTGCCTGCTTCCCGCCGCACCCCAGCCCCCCTCAAATG |
| | | CCTTCCGAGAAGCCCATTGAGCAGGGGGCTTGCATTGCACCCCAGC |
| | | CTGACAGCCTGGCATCTTGGGATAAAAGCAGCACAGCCCCCTAGGG |
| | | GCTGCCCTTGCTGTGTGGCGCCACCGGCGGTGGAGAACAAGGCTCT |
| | | ATTCAGCCTGTGCCCAGGAAAGGGGATCAGGGGATGCCCAGGCATG |
| | | GACAGTGGGTGGCAGGGGGGGAGAGGAGGGCTGTCTGCTTCCCAG |
| | | AAGTCCAAGGACACAAATGGGTGAGGGGACTGGGCAGGGTTCTGAC |
| | | CCTGTGGGACCAGAGTGGAGGGCGTAGATGGACCTGAAGTCTCCAG |
| | | GGACAACAGGGCCCAGGTCTCAGGCTCCTAGTTGGGCCCAGTGGCT |
| | | CCAGCGTTTCCAAACCCATCCATCCCCAGAGGTTCTTCCCATCTCTC |
| | | CAGGCTGATGTGTGGGAACTCGAGGAAATAAATCTCCAGTGGGAGA |
| | | CGGAGGGGTGGCCAGGGAAACGGGGCGCTGCAGGAATAAAGACGA |
| | | GCCAGCACAGCCAGCTCATGTGTAACGGCTTTGTGGAGCTGTCAAG |
| | | GCCTGGTCTCTGGGAGAGAGGCACAGGGAGGCCAGACAAGGAAGG |
| | | GGTGACCTGGAGGGACAGATCCAGGGGCTAAAGTCCTGATAAGGCA |
| | | AGAGAGTGCCGGCCCCCTCTTGCCCTATCAGGACCTCCACTGCCAC |
| | | ATAGAGGCCATGATTGACCCTTAGACAAAGGGCTGGTGTCCAATCCC |
| | | AGCCCCCAGCCCCAGAACTCCAGGGAATGAATGGGCAGAGAGCAGG |
| | | AATGTGGGACATCTGTGTTCAAGGGAAGGACTCCAGGAGTCTGCTG |
| | | GGAATGAGGCCTAGTAGGAAATGAGGTGGCCCTTGAGGGTACAGAA |
| | | CAGGTTCATTCTTCGCCAAATTCCCAGCACCTTGCAGGCACTTACAG |
| | | CTGAGTGAGATAATGCCTGGGTTATGAAATCAAAAAGTTGGAAAGCA |
| | | GGTCAGAGGTCATCTGGTACAGCCCTTCCTTCCCTTTTTTTTTTTTTTT |
| | | TTTGTGAGACAAGGTCTCTCTCTGTTGCCCAGGCTGGAGTGGCGCAA |
| | | ACACAGCTCACTGCAGCCTCAACCTACTGGGCTCAAGCAATCCTCCA |
| | | GCCTCAGCCTCCCAAAGTGCTGGGATTACAAGCATGAGCCACCCCA |
| | | CTCAGCCCTTTCCTTCCTTTTTAATTGATGCATAATAATTGTAAGTATT |
| | | CATCATGGTCCAACCAACCCTTTCTTGACCCACCTTCCTAGAGAGAG |
| | | GGTCCTCTTGCTTCAGCGGTCAGGGCCCCAGACCCATGGTCTGGCT |
| | | CCAGGTACCACCTGCCTCATGCAGGAGTTGGCGTGCCCAGGAAGCT |
| | | CTGCCTCTGGGCACAGTGACCTCAGTGGGGTGAGGGGAGCTCTCCC |
| | | CATAGCTGGGCTGCGGCCCAACCCCACCCCCTCAGGCTATGCCAGG |
| | | GGGTGTTGCCAGGGGCACCCGGGCATCGCCAGTCTAGCCCACTCCT |
| | | TCATAAAGCCCTCGCATCCCAGGAGCGAGCAGAGCCAGAGCAGGTT |
| | | GGAGAGGAGACGCATCACCTCCGCTGCTCGC |

In some embodiments, wild-type Atoh1, or a variant thereof, such as a polynucleotide sequence that encodes a protein having at least 85% sequence identity (e.g., 85%, 90%, 95%, 96%, 97%, 98%, 99%, or more, sequence identity) to the amino acid sequence of wild-type mammalian (e.g., human or mouse) Atoh1 (e.g., SEQ ID NO: 10 or SEQ ID NO: 12) is operably linked to a high expression promoter (e.g., a high expression, supporting cell-specific promoter) described herein. In some embodiments, the polynucleotide sequence encoding an Atoh1 protein encodes an amino acid sequence that contains one or more conservative amino acid substitutions relative to SEQ ID NO: 10 (e.g., 1, 2, 3, 4, 5, 6, 7, 8, 9, 10, 11, 12, 13, 14, 15, 16, 17, 18, 19, 20 or more conservative amino acid substitutions), provided that the Atoh1 analog encoded retains the therapeutic function of wild-type Atoh1 (e.g., the ability to promote hair cell development). No more than 10% of the amino acids in the Atoh1 protein may be replaced with conservative amino acid substitutions. In some embodiments, the polynucleotide sequence that encodes Atoh1 is any polynucleotide sequence that, by redundancy of the genetic code, encodes SEQ ID NO: 10. The polynucleotide sequence that encodes Atoh1 can be partially or fully codon-optimized for expression (e.g. in human supporting cells). Atoh1 may be encoded by a polynucleotide having the sequence of SEQ ID NO: 11. The Atoh1 protein may be a human Atoh1 protein or may be a homolog of the human Atoh1 protein from another mammalian species (e.g., mouse, rat, cow, horse, goat, sheep, donkey, cat, dog, rabbit, guinea pig, or other mammal). Exemplary Atoh1 amino acid and polynucleotide sequences are listed in Table 3, below.

TABLE 3
Atoh1 sequences

| SEQ | Description of | |
|---|---|---|
| ID | promoter | |
| NO: | sequence | Sequence |
| 10 | Human Atoh1 amino | MSRLLHAEEWAEVKELGDHHRQPQPHHLPQPPPPPQPPATLQARE |
| | acid sequence, | HPVYPPELSLLDSTDPRAWLAPTLQGICTARAAQYLLHSPELGASEA |
| | UniProt Q92858, | AAPRDEVDGRGELVRRSSGGASSSKSPGPVKVREQLCKLKGGVVV |
| | RefSeq accession | DELGCSRQRAPSSKQVNGVQKQRRLAANARERRRMHGLNHAFDQL |
| | number | RNVIPSFNNDKKLSKYETLQMAQIYINALSELLQTPSGGEQPPPPPAS |
| | NP_005163.1 | CKSDHHHLRTAASYEGGAGNATAAGAQQASGGSQRPTPPGSCRTR |
| | | FSAPASAGGYSVQLDALHFSTFEDSALTAMMAQKNLSPSLPGSILQP |
| | | VQEENSKTSPRSHRSDGEFSPHSHYSDSDEAS |
| 11 | Human ATOH1 | ATGTCCCGCCTGCTGCATGCAGAAGAGTGGGCTGAAGTGAAGGA |
| | protein coding | GTTGGGAGACCACCATCGCCAGCCCCAGCCGCATCATCTCCCGC |
| | sequence, also | AACCGCCGCCGCCGCCGCAGCCACCTGCAACTTTGCAGGCGAGA |
| | documented under | GAGCATCCCGTCTACCCGCCTGAGCTGTCCCTCCTGGACAGCAC |
| | RefSeq accession | CGACCCACGCGCCTGGCTGGCTCCCACTTTGCAGGGCATCTGCA |
| | number | CGGCACGCGCCGCCCAGTATTTGCTACATTCCCCGGAGCTGGGT |
| | NM_005172.2 | GCCTCAGAGGCCGCTGCGCCCCGGGACGAGGTGGACGGCCGGG |
| | | GGGAGCTGGTAAGGAGGAGCAGCGGCGGTGCCAGCAGCAGCAA |
| | | GAGCCCCGGGCCGGTGAAAGTGCGGGAACAGCTGTGCAAGCTG |
| | | AAAGGCGGGGTGGTGGTAGACGAGCTGGGCTGCAGCCGCCAAC |
| | | GGGCCCCTTCCAGCAAACAGGTGAATGGGGTGCAGAAGCAGAGA |
| | | CGGCTAGCAGCCAACGCCAGGGAGCGGCGCAGGATGCATGGGC |
| | | TGAACCACGCCTTCGACCAGCTGCGCAATGTTATCCCGTCGTTCA |
| | | ACAACGACAAGAAGCTGTCCAAATATGAGACCCTGCAGATGGCCC |
| | | AAATCTACATCAACGCCTTGTCCGAGCTGCTACAAACGCCCAGCG |
| | | GAGGGGAACAGCCACCGCCGCCTCCAGCCTCCTGCAAAAGCGAC |
| | | CACCACCACCTTCGCACCGCGGCCTCCTATGAAGGGGGCGCGGG |
| | | CAACGCGACCGCAGCTGGGGCTCAGCAGGCTTCCGGAGGGAGC |
| | | CAGCGGCCGACCCCGCCCGGGAGTTGCCGGACTCGCTTCTCAGC |
| | | CCCAGCTTCTGCGGGAGGGTACTCGGTGCAGCTGGACGCTCTGC |
| | | ACTTCTCGACTTTCGAGGACAGCGCCCTGACAGCGATGATGGCG |
| | | CAAAAGAATTTGTCTCCTTCTCTCCCCGGGAGCATCTTGCAGCCA |
| | | GTGCAGGAGGAAAACAGCAAAACTTCGCCTCGGTCCCACAGAAG |
| | | CGACGGGGATTTTCCCCCCATTCCCATTACAGTGACTCGGATGA |
| | | GGCAAGT |
| 12 | Murine Atoh1 amino | MSRLLHAEEWAEVKELGDHHRHPQPHHVPPLTPQPPATLQARDLPV |
| | acid sequence, | YPAELSLLDSTDPRAWLTPTLQGLCTARAAQYLLHSPELGASEAAAP |
| | UniProt P48985 | RDEADSQGELVRRSGCGGLSKSPGPVKVREQLCKLKGGVVVDELG |
| | | CSRQRAPSSKQVNGVQKQRRLAANARERRRMHGLNHAFDQLRNVI |
| | | PSFNNDKKLSKYETLQMAQIYINALSELLQTPNVGEQPPPPTASCKN |
| | | DHHHLRTASSYEGGAGASAVAGAQPAPGGGPRPTPPGPCRTRFSG |
| | | PASSGGYSVQLDALHFPAFEDRALTAMMAQKDLSPSLPGGILQPVQ |
| | | EDNSKTSPRSHRSDGEFSPHSHYSDSDEAS |
| 13 | Murine ATOH1 | ATGTCCCGCCTGCTGCATGCAGAAGAGTGGGCTGAGGTAAAAGA |
| | protein coding | GTTGGGGGACCACCATCGCCATCCCCAGCCGCACCACGTCCCGC |
| | sequence, also | CGCTGACGCCACAGCCACCTGCTACCCTGCAGGCGAGAGACCTT |
| | documented under | CCCGTCTACCCGGCAGAACTGTCCCTCCTGGATAGCACCGACCC |
| | RefSeq accession | ACGCGCCTGGCTGACTCCCACTTTGCAGGGCCTCTGCACGGCAC |
| | number | GCGCCGCCCAGTATCTGCTGCATTCTCCCGAGCTGGGTGCCTCC |
| | NM_007500.5 | GAGGCCGCGGCGCCCCGGGACGAGGCTGACAGCCAGGGTGAGC |
| | | TGGTAAGGAGAAGCGGCTGTGGCGGCCTCAGCAAGAGCCCCGG |
| | | GCCCGTCAAAGTACGGGAACAGCTGTGCAAGCTGAAGGGTGGGG |
| | | TTGTAGTGGACGAGCTTGGCTGCAGCCGCCAGCGAGCCCCTTCC |
| | | AGCAAACAGGTGAATGGGGTACAGAAGCAAAGGAGGCTGGCAGC |
| | | AAACGCAAGGGAACGGCGCAGGATGCACGGGCTGAACCACGCCT |
| | | TCGACCAGCTGCGCAACGTTATCCCGTCCTTCAACAACGACAAGA |
| | | AGCTGTCCAAATATGAGACCCTACAGATGGCCCAGATCTACATCA |
| | | ACGCTCTGTCGGAGTTGCTGCAGACTCCCAATGTCGGAGAGCAA |
| | | CCGCCGCCGCCCACAGCTTCCTGCAAAAATGACCACCATCACCTT |
| | | CGCACCGCCTCCTCCTATGAAGGAGGTGCGGGCGCCTCTGCGGT |
| | | AGCTGGGGCTCAGCCAGCCCCGGGAGGGGGCCCGAGACCTACC |
| | | CCGCCCGGGCCTTGCCGGACTCGCTTCTCAGGCCCAGCTTCCTC |
| | | TGGGGGTTACTCGGTGCAGCTGGACGCTTTGCACTTCCCAGCCTT |
| | | CGAGGACAGGGCCCTAACAGCGATGATGGCACAGAAGGACCTGT |
| | | CGCCTTCGCTGCCCGGGGGCATCCTGCAGCCTGTACAGGAGGAC |
| | | AACAGCAAAACATCTCCCAGATCCCACAGAAGTGACGGAGAGTTT |
| | | TCCCCCCACTCTCATTACAGTGACTCTGATGAGGCCAGT |

Transfer plasmids that may be used to produce nucleic acid vectors (e.g., AAV vectors) for use in the compositions and methods described herein are provided in Table 4. A transfer plasmid (e.g., a plasmid containing a DNA sequence to be delivered by a nucleic acid vector, e.g., to be delivered by an AAV) may be co-delivered into producer cells with a helper plasmid (e.g., a plasmid providing proteins necessary for AAV manufacture) and a rep/cap plasmid (e.g., a plasmid that provides AAV capsid proteins and proteins that insert the transfer plasmid DNA sequence into the capsid shell) to produce a nucleic acid vector (e.g., an AAV vector) for administration. The transfer plasmids provided in Table 4 can be used to produce nucleic acid vectors (e.g., AAV vectors) containing a high expression, supporting cell specific promoter (a GFAP promoter having the sequence of SEQ ID NO: 8) operably linked to a polynucleotide encoding Atoh1.

TABLE 4
Transgene Plasmid sequences

| SEQ ID | Description of | |
|---|---|---|
| NO: | plasmid sequence | Sequence |
| 14 | short GFAP-hAtoh1- | CTGCGCGCTCGCTCGCTCACTGAGGCCGCCCGGGCAAAGCCC |
| | 2A-H2BGFP | GGGCGTCGGGCGACCTTTGGTCGCCCGGCCTCAGTGAGCGAG |
| | First (5′) ITR at | CGAGCGCGCAGAGAGGGAGTGGCCAACTCCATCACTAGGGGTT |
| | positions 1-130 | CCTTGTAGTTAATGATTAACCCGCCATGCTACTTATCTACGTAGC |
| | GFAP promoter at | CATGCTCTAGGAAGATCGGAATTCGCCCTTAAGCTAGCGGCGC |
| | positions 228-908 | GCCACACCGGTGAACATATCCTGGTGTGGAGTAGGGGACGCTG |
| | ATOH 1 coding | CTCTGACAGAGGCTCGGGGGCCTGAGCTGGCTCTGTGAGCTGG |
| | sequence at | GGAGGAGGCAGACAGCCAGGCCTTGTCTGCAAGCAGACCTGGC |
| | positions 925-1986 | AGCATTGGGCTGGCCGCCCCCCAGGGCCTCCTCTTCATGCCCA |
| | P2A at positions | GTGAATGACTCACCTTGGCACAGACACAATGTTCGGGGTGGGC |
| | 2002-2058 | ACAGTGCCTGCTTCCCGCCGCACCCCAGCCCCCCTCAAATGCC |
| | H2B-GFP sequence | TTCCGAGAAGCCCATTGAGCAGGGGGCTTGCATTGCACCCCAG |
| | at positions 2059- | CCTGACAGCCTGGCATCTTGGGATAAAAGCAGCACAGCCCCCT |
| | 3174 | AGGGGCTGCCCTTGCTGTGTGGCGCCACCGGCGGTGGAGAAC |
| | WPRE at positions | AAGGCTCTATTCAGCCTGTGCCCAGGAAAGGGGATCAGGGGAT |
| | 3183-3730 | GCCCAGGCATGGACAGTGGGTGGCAGGGGGGGAGAGGAGGG |
| | bGH polyA signal at | CTGTCTGCTTCCCAGAAGTCCAAGGACACAAATGGGTGAGGGG |
| | positions 3743-3950 | AGAGCTCTCCCCATAGCTGGGCTGCGGCCCAACCCCACCCCCT |
| | Second (3′) ITR at | CAGGCTATGCCAGGGGGTGTTGCCAGGGGCACCCGGGCATCG |
| | positions 4038-4167 | CCAGTCTAGCCCACTCCTTCATAAAGCCCTCGCATCCCAGGAGC |
| | Transgene to be | GAGCAGAGCCAGAGCAGGTTGGAGAGGAGACGCATCACCTCCG |
| | transferred into | CTGCTCGCCCGCGGCCGCGCCACCATGTCCCGCCTGCTGCATG |
| | vector at positions 1- | CAGAAGAGTGGGCTGAAGTGAAGGAGTTGGGAGACCACCATCG |
| | 4167 | CCAGCCCCAGCCGCATCATCTCCCGCAACCGCCGCCGCCGCC |
| | | GCAGCCACCTGCAACTTTGCAGGCGAGAGAGCATCCCGTCTAC |
| | | CCGCCTGAGCTGTCCCTCCTGGACAGCACCGACCCACGCGCCT |
| | | GGCTGGCTCCCACTTTGCAGGGCATCTGCACGGCACGCGCCGC |
| | | CCAGTATTTGCTACATTCCCCGGAGCTGGGTGCCTCAGAGGCC |
| | | GCTGCGCCCCGGGACGAGGTGGACGGCCGGGGGGAGCTGGTA |
| | | AGGAGGAGCAGCGGCGGTGCCAGCAGCAGCAAGAGCCCCGGG |
| | | CCGGTGAAAGTGCGGGAACAGCTGTGCAAGCTGAAAGGCGGG |
| | | GTGGTGGTAGACGAGCTGGGCTGCAGCCGCCAACGGGCCCCT |
| | | TCCAGCAAACAGGTGAATGGGGTGCAGAAGCAGAGACGGCTAG |
| | | CAGCCAACGCCAGGGAGCGGCGCAGGATGCATGGGCTGAACC |
| | | ACGCCTTCGACCAGCTGCGCAATGTTATCCCGTCGTTCAACAAC |
| | | GACAAGAAGCTGTCCAAATATGAGACCCTGCAGATGGCCCAAAT |
| | | CTACATCAACGCCTTGTCCGAGCTGCTACAAACGCCCAGCGGA |
| | | GGGGAACAGCCACCGCCGCCTCCAGCCTCCTGCAAAAGCGACC |
| | | ACCACCACCTTCGCACCGCGGCCTCCTATGAAGGGGGCGCGG |
| | | GCAACGCGACCGCAGCTGGGGCTCAGCAGGCTTCCGGAGGGA |
| | | GCCAGCGGCCGACCCCGCCCGGGAGTTGCCGGACTCGCTTCT |
| | | CAGCCCCAGCTTCTGCGGGAGGGTACTCGGTGCAGCTGGACGC |
| | | TCTGCACTTCTCGACTTTCGAGGACAGCGCCCTGACAGCGATGA |
| | | TGGCGCAAAAGAATTTGTCTCCTTCTCTCCCCGGGAGCATCTTG |
| | | CAGCCAGTGCAGGAGGAAAACAGCAAAACTTCGCCTCGGTCCC |
| | | ACAGAAGCGACGGGGAATTTTCCCCCCATTCCCATTACAGTGAC |
| | | TCGGATGAGGCAAGTACGCGTGGAAGCGGAGCTACTAACTTCA |
| | | GCCTGCTGAAGCAGGCTGGCGACGTGGAGGAGAACCCTGGAC |
| | | CTATGCCAGAGCCAGCGAAGTCTGCTCCCGCCCCGAAAAAGGG |
| | | CTCCAAGAAGGCGGTGACTAAGGCGCAGAAGAAAGGCGGCAAG |
| | | AAGCGCAAGCGCAGCCGCAAGGAGAGCTATTCCATCTATGTGTA |
| | | CAAGGTTCTGAAGCAGGTCCACCCTGACACCGGCATTTCGTCCA |
| | | AGGCCATGGGCATCATGAATTCGTTTGTGAACGACATTTTCGAG |
| | | CGCATCGCAGGTGAGGCTTCCCGCCTGGCGCATTACAACAAGC |
| | | GCTCGACCATCACCTCCAGGGAGATCCAGACGGCCGTGCGCCT |
| | | GCTGCTGCCTGGGGAGTTGGCCAAGCACGCCGTGTCCGAGGG |
| | | TACTAAGGCCATCACCAAGTACACCAGCGCTAAGGATCCACCGG |
| | | TCGCCACCATGGTGAGCAAGGGCGAGGAGCTGTTCACCGGGGT |
| | | GGTGCCCATCCTGGTCGAGCTGGACGGCGACGTAAACGGCCAC |
| | | AAGTTCAGCGTGTCCGGCGAGGGCGAGGGCGATGCCACCTAC |
| | | GGCAAGCTGACCCTGAAGTTCATCTGCACCACCGGCAAGCTGC |
| | | CCGTGCCCTGGCCCACCCTCGTGACCACCCTGACCTACGGCGT |
| | | GCAGTGCTTCAGCCGCTACCCCGACCACATGAAGCAGCACGAC |
| | | TTCTTCAAGTCCGCCATGCCCGAAGGCTACGTCCAGGAGCGCA |
| | | CCATCTTCTTCAAGGACGACGGCAACTACAAGACCCGCGCCGA |
| | | GGTGAAGTTCGAGGGCGACACCCTGGTGAACCGCATCGAGCTG |
| | | AAGGGCATCGACTTCAAGGAGGACGGCAACATCCTGGGGCACA |
| | | AGCTGGAGTACAACTACAACAGCCACAACGTCTATATCATGGCC |
| | | GACAAGCAGAAGAACGGCATCAAGGTGAACTTCAAGATCCGCC |
| | | ACAACATCGAGGACGGCAGCGTGCAGCTCGCCGACCACTACCA |
| | | GCAGAACACCCCCATCGGCGACGGCCCCGTGCTGCTGCCCGA |
| | | CAACCACTACCTGAGCACCCAGTCCGCCCTGAGCAAAGACCCC |
| | | AACGAGAAGCGCGATCACATGGTCCTGCTGGAGTTCGTGACCG |
| | | CCGCCGGGATCACTCTCGGCATGGACGAGCTGTACAAGTAATA |
| | | AGCTTGGATCCAATCAACCTCTGGATTACAAAATTTGTGAAAGAT |
| | | TGACTGGTATTCTTAACTATGTTGCTCCTTTTACGCTATGTGGAT |
| | | ACGCTGCTTTAATGCCTTTGTATCATGCTATTGCTTCCCGTATGG |
| | | CTTTCATTTTCTCCTCCTTGTATAAATCCTGGTTGCTGTCTCTTTA |
| | | TGAGGAGTTGTGGCCCGTTGTCAGGCAACGTGGCGTGGTGTGC |
| | | ACTGTGTTTGCTGACGCAACCCCCACTGGTTGGGGCATTGCCAC |
| | | CACCTGTCAGCTCCTTTCCGGGACTTTCGCTTTCCCCCTCCCTA |
| | | TTGCCACGGCGGAACTCATCGCCGCCTGCCTTGCCCGCTGCTG |
| | | GACAGGGGCTCGGCTGTTGGGCACTGACAATTCCGTGGTGTTG |
| | | TCGGGGAAATCATCGTCCTTTCCTTGGCTGCTCGCCTGTGTTGC |
| | | CACCTGGATTCTGCGCGGGACGTCCTTCTGCTACGTCCCTTCG |
| | | GCCCTCAATCCAGCGGACCTTCCTTCCCGCGGCCTGCTGCCGG |
| | | CTCTGCGGCCTCTTCCGCGTCTTCGAGATCTGCCTCGACTGTGC |
| | | CTTCTAGTTGCCAGCCATCTGTTGTTTGCCCCTCCCCCGTGCCT |
| | | TCCTTGACCCTGGAAGGTGCCACTCCCACTGTCCTTTCCTAATA |
| | | AAATGAGGAAATTGCATCGCATTGTCTGAGTAGGTGTCATTCTAT |
| | | TCTGGGGGGTGGGGTGGGGCAGGACAGCAAGGGGGAGGATTG |
| | | GGAAGACAATAGCAGGCATGCTGGGGACTCGAGTTAAGGGCGA |
| | | ATTCCCGATAAGGATCTTCCTAGAGCATGGCTACGTAGATAAGT |
| | | AGCATGGCGGGTTAATCATTAACTACAAGGAACCCCTAGTGATG |
| | | GAGTTGGCCACTCCCTCTCTGCGCGCTCGCTCGCTCACTGAGG |
| | | CCGGGCGACCAAAGGTCGCCCGACGCCCGGGCTTTGCCCGGG |
| | | CGGCCTCAGTGAGCGAGCGAGCGCGCAGCCTTAATTAACCTAA |
| | | TTCACTGGCCGTCGTTTTACAACGTCGTGACTGGGAAAACCCTG |
| | | GCGTTACCCAACTTAATCGCCTTGCAGCACATCCCCCTTTCGCC |
| | | AGCTGGCGTAATAGCGAAGAGGCCCGCACCGATCGCCCTTCCC |
| | | AACAGTTGCGCAGCCTGAATGGCGAATGGGACGCGCCCTGTAG |
| | | CGGCGCATTAAGCGCGGCGGGTGTGGTGGTTACGCGCAGCGT |
| | | GACCGCTACACTTGCCAGCGCCCTAGCGCCCGCTCCTTTCGCT |
| | | TTCTTCCCTTCCTTTCTCGCCACGTTCGCCGGCTTTCCCCGTCA |
| | | AGCTCTAAATCGGGGGCTCCCTTTAGGGTTCCGATTTAGTGCTT |
| | | TACGGCACCTCGACCCCAAAAAACTTGATTAGGGTGATGGTTCA |
| | | CGTAGTGGGCCATCGCCCTGATAGACGGTTTTTCGCCCTTTGAC |
| | | GTTGGAGTCCACGTTCTTTAATAGTGGACTCTTGTTCCAAACTGG |
| | | AACAACACTCAACCCTATCTCGGTCTATTCTTTTGATTTATAAGG |
| | | GATTTTGCCGATTTCGGCCTATTGGTTAAAAAATGAGCTGATTTA |
| | | ACAAAAATTTAACGCGAATTTTAACAAAATATTAACGCTTACAATT |
| | | TAGGTGGCACTTTTCGGGGAAATGTGCGCGGAACCCCTATTTGT |
| | | TTATTTTTCTAAATACATTCAAATATGTATCCGCTCATGAGACAAT |
| | | AACCCTGATAAATGCTTCAATAATATTGAAAAAGGAAGAGTATGA |
| | | GTATTCAACATTTCCGTGTCGCCCTTATTCCCTTTTTTGCGGCAT |
| | | TTTGCCTTCCTGTTTTTGCTCACCCAGAAACGCTGGTGAAAGTAA |
| | | AAGATGCTGAAGATCAGTTGGGTGCACGAGTGGGTTACATCGAA |
| | | CTGGATCTCAACAGCGGTAAGATCCTTGAGAGTTTTCGCCCCGA |
| | | AGAACGTTTTCCAATGATGAGCACTTTTAAAGTTCTGCTATGTGG |
| | | CGCGGTATTATCCCGTATTGACGCCGGGCAAGAGCAACTCGGT |
| | | CGCCGCATACACTATTCTCAGAATGACTTGGTTGAGTACTCACC |
| | | AGTCACAGAAAAGCATCTTACGGATGGCATGACAGTAAGAGAAT |
| | | TATGCAGTGCTGCCATAACCATGAGTGATAACACTGCGGCCAAC |
| | | TTACTTCTGACAACGATCGGAGGACCGAAGGAGCTAACCGCTTT |
| | | TTTGCACAACATGGGGGATCATGTAACTCGCCTTGATCGTTGGG |
| | | AACCGGAGCTGAATGAAGCCATACCAAACGACGAGCGTGACAC |
| | | CACGATGCCTGTAGCAATGGCAACAACGTTGCGCAAACTATTAA |
| | | CTGGCGAACTACTTACTCTAGCTTCCCGGCAACAATTAATAGACT |
| | | GGATGGAGGCGGATAAAGTTGCAGGACCACTTCTGCGCTCGGC |
| | | CCTTCCGGCTGGCTGGTTTATTGCTGATAAATCTGGAGCCGGTG |
| | | AGCGTGGGTCTCGCGGTATCATTGCAGCACTGGGGCCAGATGG |
| | | TAAGCCCTCCCGTATCGTAGTTATCTACACGACGGGGAGTCAGG |
| | | CAACTATGGATGAACGAAATAGACAGATCGCTGAGATAGGTGCC |
| | | TCACTGATTAAGCATTGGTAACTGTCAGACCAAGTTTACTCATAT |
| | | ATACTTTAGATTGATTTAAAACTTCATTTTTAATTTAAAAGGATCTA |
| | | GGTGAAGATCCTTTTTGATAATCTCATGACCAAAATCCCTTAACG |
| | | TGAGTTTiCGTTCCACTGAGCGTCAGACCCCGTAGAAAAGATCA |
| | | AAGGATCTTCTTGAGATCCTTTTTTTCTGCGCGTAATCTGCTGCT |
| | | TGCAAACAAAAAAACCACCGCTACCAGCGGTGGTTTGTTTGCCG |
| | | GATCAAGAGCTACCAACTCTTTTTCCGAAGGTAACTGGCTTCAG |
| | | CAGAGCGCAGATACCAAATACTGTTCTTCTAGTGTAGCCGTAGT |
| | | TAGGCCACCACTTCAAGAACTCTGTAGCACCGCCTACATACCTC |
| | | GCTCTGCTAATCCTGTTACCAGTGGCTGCTGCCAGTGGCGATAA |
| | | GTCGTGTCTTACCGGGTTGGACTCAAGACGATAGTTACCGGATA |
| | | AGGCGCAGCGGTCGGGCTGAACGGGGGGTTCGTGCACACAGC |
| | | CCAGCTTGGAGCGAACGACCTACACCGAACTGAGATACCTACA |
| | | GCGTGAGCTATGAGAAAGCGCCACGCTTCCCGAAGGGAGAAAG |
| | | GCGGACAGGTATCCGGTAAGCGGCAGGGTCGGAACAGGAGAG |
| | | CGCACGAGGGAGCTTCCAGGGGGAAACGCCTGGTATCTTTATA |
| | | GTCCTGTCGGGTTTCGCCACCTCTGACTTGAGCGTCGATTTTTG |
| | | TGATGCTCGTCAGGGGGGCGGAGCCTATGGAAAAACGCCAGCA |
| | | ACGCGGCCTTTTTACGGTTCCTGGCCTTTTGCTGGCCTTTTGCT |
| | | CACATGTTCTTTCCTGCGTTATCCCCTGATTCTGTGGATAACCGT |
| | | ATTACCGCCTTTGAGTGAGCTGATACCGCTCGCCGCAGCCGAA |
| | | CGACCGAGCGCAGCGAGTCAGTGAGCGAGGAAGCGGAAGAGC |
| | | GCCCAATACGCAAACCGCCTCTCCCCGCGCGTTGGCCGATTCA |
| | | TTAATGCAGCTGGCACGACAGGTTTCCCGACTGGAAAGCGGGC |
| | | AGTGAGCGCAACGCAATTAATGTGAGTTAGCTCACTCATTAGGC |
| | | ACCCCAGGCTTTACACTTTATGCTTCCGGCTCGTATGTTGTGTG |
| | | GAATTGTGAGCGGATAACAATTTCACACAGGAAACAGCTATGAC |
| | | CATGATTACGCCAGATTTAATTAAGGCCTTAATTAGG |
| 15 | short GFAP-hAtoh1 | CTGCGCGCTCGCTCGCTCACTGAGGCCGCCCGGGCAAAGCCC |
| | | GGGCGTCGGGCGACCTTTGGTCGCCCGGCCTCAGTGAGCGAG |
| | First (5′) ITR at | CGAGCGCGCAGAGAGGGAGTGGCCAACTCCATCACTAGGGGTT |
| | positions 1-130 | CCTTGTAGTTAATGATTAACCCGCCATGCTACTTATCTACGTAGC |
| | GFAP promoter at | CATGCTCTAGGAAGATCGGAATTCGCCCTTAAGCTAGCGGCGC |
| | positions 228-908 | GCCACACCGGTGAACATATCCTGGTGTGGAGTAGGGGACGCTG |
| | ATOH1 coding | CTCTGACAGAGGCTCGGGGGCCTGAGCTGGCTCTGTGAGCTGG |
| | sequence at | GGAGGAGGCAGACAGCCAGGCCTTGTCTGCAAGCAGACCTGGC |
| | positions 925-1986 | AGCATTGGGCTGGCCGCCCCCCAGGGCCTCCTCTTCATGCCCA |
| | WPRE at positions | GTGAATGACTCACCTTGGCACAGACACAATGTTCGGGGTGGGC |
| | 1997-2544 | ACAGTGCCTGCTTCCCGCCGCACCCCAGCCCCCCTCAAATGCC |
| | bGH polyA signal at | TTCCGAGAAGCCCATTGAGCAGGGGGCTTGCATTGCACCCCAG |
| | positions 2557-2764 | CCTGACAGCCTGGCATCTTGGGATAAAAGCAGCACAGCCCCCT |
| | Second (3′) ITR at | AGGGGCTGCCCTTGCTGTGTGGCGCCACCGGCGGTGGAGAAC |
| | positions 2852-2981 | AAGGCTCTATTCAGCCTGTGCCCAGGAAAGGGGATCAGGGGAT |
| | Transgene to be | GCCCAGGCATGGACAGTGGGTGGCAGGGGGGGAGAGGAGGG |
| | transferred into | CTGTCTGCTTCCCAGAAGTCCAAGGACACAAATGGGTGAGGGG |
| | vector at positions | AGAGCTCTCCCCATAGCTGGGCTGCGGCCCAACCCCACCCCCT |
| | 1-2981 | CAGGCTATGCCAGGGGGTGTTGCCAGGGGCACCCGGGCATCG |
| | | CCAGTCTAGCCCACTCCTTCATAAAGCCCTCGCATCCCAGGAGC |
| | | GAGCAGAGCCAGAGCAGGTTGGAGAGGAGACGCATCACCTCCG |
| | | CTGCTCGCCCGCGGCCGCGCCACCATGTCCCGCCTGCTGCATG |
| | | CAGAAGAGTGGGCTGAAGTGAAGGAGTTGGGAGACCACCATCG |
| | | CCAGCCCCAGCCGCATCATCTCCCGCAACCGCCGCCGCCGCC |
| | | GCAGCCACCTGCAACTTTGCAGGCGAGAGAGCATCCCGTCTAC |
| | | CCGCCTGAGCTGTCCCTCCTGGACAGCACCGACCCACGCGCCT |
| | | GGCTGGCTCCCACTTTGCAGGGCATCTGCACGGCACGCGCCGC |
| | | CCAGTATTTGCTACATTCCCCGGAGCTGGGTGCCTCAGAGGCC |
| | | GCTGCGCCCCGGGACGAGGTGGACGGCCGGGGGGAGCTGGTA |
| | | AGGAGGAGCAGCGGCGGTGCCAGCAGCAGCAAGAGCCCCGGG |
| | | CCGGTGAAAGTGCGGGAACAGCTGTGCAAGCTGAAAGGCGGG |
| | | GTGGTGGTAGACGAGCTGGGCTGCAGCCGCCAACGGGCCCCT |
| | | TCCAGCAAACAGGTGAATGGGGTGCAGAAGCAGAGACGGCTAG |
| | | CAGCCAACGCCAGGGAGCGGCGCAGGATGCATGGGCTGAACC |
| | | ACGCCTTCGACCAGCTGCGCAATGTTATCCCGTCGTTCAACAAC |
| | | GACAAGAAGCTGTCCAAATATGAGACCCTGCAGATGGCCCAAAT |
| | | CTACATCAACGCCTTGTCCGAGCTGCTACAAACGCCCAGCGGA |
| | | GGGGAACAGCCACCGCCGCCTCCAGCCTCCTGCAAAAGCGACC |
| | | ACCACCACCTTCGCACCGCGGCCTCCTATGAAGGGGGCGCGG |
| | | GCAACGCGACCGCAGCTGGGGCTCAGCAGGCTTCCGGAGGGA |
| | | GCCAGCGGCCGACCCCGCCCGGGAGTTGCCGGACTCGCTTCT |
| | | CAGCCCCAGCTTCTGCGGGAGGGTACTCGGTGCAGCTGGACGC |
| | | TCTGCACTTCTCGACTTTCGAGGACAGCGCCCTGACAGCGATGA |
| | | TGGCGCAAAAGAATTTGTCTCCTTCTCTCCCCGGGAGCATCTTG |
| | | CAGCCAGTGCAGGAGGAAAACAGCAAAACTTCGCCTCGGTCCC |
| | | ACAGAAGCGACGGGGAATTTTCCCCCCATTCCCATTACAGTGAC |
| | | TCGGATGAGGCAAGTTAGAAGCTTGGATCCAATCAACCTCTGGA |
| | | TTACAAAATTTGTGAAAGATTGACTGGTATTCTTAACTATGTTGCT |
| | | CCTTTTACGCTATGTGGATACGCTGCTTTAATGCCTTTGTATCAT |
| | | GCTATTGCTTCCCGTATGGCTTTCATTTTCTCCTCCTTGTATAAAT |
| | | CCTGGTTGCTGTCTCTTTATGAGGAGTTGTGGCCCGTTGTCAGG |
| | | CAACGTGGCGTGGTGTGCACTGTGTTTGCTGACGCAACCCCCA |
| | | CTGGTTGGGGCATTGCCACCACCTGTCAGCTCCTTTCCGGGACT |
| | | TTCGCTTTCCCCCTCCCTATTGCCACGGCGGAACTCATCGCCGC |
| | | CTGCCTTGCCCGCTGCTGGACAGGGGCTCGGCTGTTGGGCACT |
| | | GACAATTCCGTGGTGTTGTCGGGGAAATCATCGTCCTTTCCTTG |
| | | GCTGCTCGCCTGTGTTGCCACCTGGATTCTGCGCGGGACGTCC |
| | | TTCTGCTACGTCCCTTCGGCCCTCAATCCAGCGGACCTTCCTTC |
| | | CCGCGGCCTGCTGCCGGCTCTGCGGCCTCTTCCGCGTCTTCGA |
| | | GATCTGCCTCGACTGTGCCTTCTAGTTGCCAGCCATCTGTTGTT |
| | | TGCCCCTCCCCCGTGCCTTCCTTGACCCTGGAAGGTGCCACTC |
| | | CCACTGTCCTTTCCTAATAAAATGAGGAAATTGCATCGCATTGTC |
| | | TGAGTAGGTGTCATTCTATTCTGGGGGGTGGGGTGGGGCAGGA |
| | | CAGCAAGGGGGAGGATTGGGAAGACAATAGCAGGCATGCTGGG |
| | | GACTCGAGTTAAGGGCGAATTCCCGATAAGGATCTTCCTAGAGC |
| | | ATGGCTACGTAGATAAGTAGCATGGCGGGTTAATCATTAACTAC |
| | | AAGGAACCCCTAGTGATGGAGTTGGCCACTCCCTCTCTGCGCG |
| | | CTCGCTCGCTCACTGAGGCCGGGCGACCAAAGGTCGCCCGAC |
| | | GCCCGGGCTTTGCCCGGGCGGCCTCAGTGAGCGAGCGAGCGC |
| | | GCAGCCTTAATTAACCTAATTCACTGGCCGTCGTTTTACAACGTC |
| | | GTGACTGGGAAAACCCTGGCGTTACCCAACTTAATCGCCTTGCA |
| | | GCACATCCCCCTTTCGCCAGCTGGCGTAATAGCGAAGAGGCCC |
| | | GCACCGATCGCCCTTCCCAACAGTTGCGCAGCCTGAATGGCGA |
| | | ATGGGACGCGCCCTGTAGCGGCGCATTAAGCGCGGCGGGTGT |
| | | GGTGGTTACGCGCAGCGTGACCGCTACACTTGCCAGCGCCCTA |
| | | GCGCCCGCTCCTTTCGCTTTCTTCCCTTCCTTTCTCGCCACGTT |
| | | CGCCGGCTTTCCCCGTCAAGCTCTAAATCGGGGGCTCCCTTTAG |
| | | GGTTCCGATTTAGTGCTTTACGGCACCTCGACCCCAAAAAACTT |
| | | GATTAGGGTGATGGTTCACGTAGTGGGCCATCGCCCTGATAGA |
| | | CGGTTTTTCGCCCTTTGACGTTGGAGTCCACGTTCTTTAATAGTG |
| | | GACTCTTGTTCCAAACTGGAACAACACTCAACCCTATCTCGGTCT |
| | | ATTCTTTTGATTTATAAGGGATTTTGCCGATTTCGGCCTATTGGT |
| | | TAAAAAATGAGCTGATTTAACAAAAATTTAACGCGAATTTTAACAA |
| | | AATATTAACGCTTACAATTTAGGTGGCACTTTTCGGGGAAATGTG |
| | | CGCGGAACCCCTATTTGTTTATTTTTCTAAATACATTCAAATATGT |
| | | ATCCGCTCATGAGACAATAACCCTGATAAATGCTTCAATAATATT |
| | | GAAAAAGGAAGAGTATGAGCCATATTCAACGGGAAACGTCGAG |
| | | GCCGCGATTAAATTCCAACATGGATGCTGATTTATATGGGTATAA |
| | | ATGGGCTCGCGATAATGTCGGGCAATCAGGTGCGACAATCTATC |
| | | GCTTGTATGGGAAGCCCGATGCGCCAGAGTTGTTTCTGAAACAT |
| | | GGCAAAGGTAGCGTTGCCAATGATGTTACAGATGAGATGGTCAG |
| | | ACTAAACTGGCTGACGGAATTTATGCCTCTTCCGACCATCAAGC |
| | | ATTiTATCCGTACTCCTGATGATGCATGGTTACTCACCACTGCGA |
| | | TCCCCGGAAAAACAGCATTCCAGGTATTAGAAGAATATCCTGATT |
| | | CAGGTGAAAATATTGTTGATGCGCTGGCAGTGTTCCTGCGCCGG |
| | | TTGCATTCGATTCCTGTTTGTAATTGTCCTTTTAACAGCGATCGC |
| | | GTATTTCGTCTTGCTCAGGCGCAATCACGAATGAATAACGGTTT |
| | | GGTTGATGCGAGTGATTTTGATGACGAGCGTAATGGCTGGCCTG |
| | | TTGAACAAGTCTGGAAAGAAATGCATAAACTTTTGCCATTCTCAC |
| | | CGGATTCAGTCGTCACTCATGGTGATTTCTCACTTGATAACCTTA |
| | | TTTTTGACGAGGGGAAATTAATAGGTTGTATTGATGTTGGACGA |
| | | GTCGGAATCGCAGACCGATACCAGGATCTTGCCATCCTATGGAA |
| | | CTGCCTCGGTGAGTTTTCTCCTTCATTACAGAAACGGCTTTTTCA |
| | | AAAATATGGTATTGATAATCCTGATATGAATAAATTGCAGTTTCAT |
| | | TTGATGCTCGATGAGTTTTTCTAACTGTCAGACCAAGTTTACTCA |
| | | TATATACTTTAGATTGATTTAAAACTTCATTTTTAATTTAAAAGGAT |
| | | CTAGGTGAAGATCCTTTTTGATAATCTCATGACCAAAATCCCTTA |
| | | ACGTGAGTTTTCGTTCCACTGAGCGTCAGACCCCGTAGAAAAGA |
| | | TCAAAGGATCTTCTTGAGATCCTTTTTTTCTGCGCGTAATCTGCT |
| | | GCTTGCAAACAAAAAAACCACCGCTACCAGCGGTGGTTTGTTTG |
| | | CCGGATCAAGAGCTACCAACTCTTTTTCCGAAGGTAACTGGCTT |
| | | CAGCAGAGCGCAGATACCAAATACTGTTCTTCTAGTGTAGCCGT |
| | | AGTTAGGCCACCACTTCAAGAACTCTGTAGCACCGCCTACATAC |
| | | CTCGCTCTGCTAATCCTGTTACCAGTGGCTGCTGCCAGTGGCGA |
| | | TAAGTCGTGTCTTACCGGGTTGGACTCAAGACGATAGTTACCGG |
| | | ATAAGGCGCAGCGGTCGGGCTGAACGGGGGGTTCGTGCACAC |
| | | AGCCCAGCTTGGAGCGAACGACCTACACCGAACTGAGATACCT |
| | | ACAGCGTGAGCTATGAGAAAGCGCCACGCTTCCCGAAGGGAGA |
| | | AAGGCGGACAGGTATCCGGTAAGCGGCAGGGTCGGAACAGGA |
| | | GAGCGCACGAGGGAGCTTCCAGGGGGAAACGCCTGGTATCTTT |
| | | ATAGTCCTGTCGGGTTTCGCCACCTCTGACTTGAGCGTCGATTT |
| | | TTGTGATGCTCGTCAGGGGGGCGGAGCCTATGGAAAAACGCCA |
| | | GCAACGCGGCCTTTTTACGGTTCCTGGCCTTTTGCTGGCCTTTT |
| | | GCTCACATGTTCTTTCCTGCGTTATCCCCTGATTCTGTGGATAAC |
| | | CGTATTACCGCCTTTGAGTGAGCTGATACCGCTCGCCGCAGCC |
| | | GAACGACCGAGCGCAGCGAGTCAGTGAGCGAGGAAGCGGAAG |
| | | AGCGCCCAATACGCAAACCGCCTCTCCCCGCGCGTTGGCCGAT |
| | | TCATTAATGCAGCTGGCACGACAGGTTTCCCGACTGGAAAGCG |
| | | GGCAGTGAGCGCAACGCAATTAATGTGAGTTAGCTCACTCATTA |
| | | GGCACCCCAGGCTTTACACTTTATGCTTCCGGCTCGTATGTTGT |
| | | GTGGAATTGTGAGCGGATAACAATTTCACACAGGAAACAGCTAT |
| | | GACCATGATTACGCCAGATTTAATTAAGGCCTTAATTAGG |

### Method for Identifying a High Expression Promoter

Suitable promoters for use in the compositions and methods described herein are high expression promoters that can induce high levels of Atoh1 expression in supporting cells. High expression promoters can be identified using the method described below.

### Adult Mouse Utricle Explant Culture and Viral Transduction

Utricles are dissected from male C57Bl/6J mice (6-8-week-old) and cultured free floating in 100 μL of base medium made up of DMEM/F12 with 5% FBS and 2.5 μg/mL ciprofloxacin at 37° C. and 5% CO₂. Gentamicin (0.5 mg/mL) is added to the medium for 24 hours to kill hair cells, after which the gentamicin is washed out and replaced with 250 μL fresh medium containing AAVs encoding Atoh1 under the control of the promoters to be tested. An AAV encoding Atoh1 under the control of the long GFAP promoter (SEQ ID NO: 9) is tested as a comparator reference. Each virus to be compared is added to a separate culture dish at an equal dose of 1 E12 vg. After one day of incubation, virus is washed out and utricles are cultured for an additional seven days in 2 mL of fresh medium (9 days total culture time).

### Cell Dissociation of Adult Mouse Utricle Explants for Single Cell RNA-Seq

Utricle explants are removed from the culture medium and incubated with EBSS containing 20 units of papain (Worthington Biochemical), 1 mM L-cysteine, 0.5 mM EDTA, 15 mM HEPES, and 15 kunitz/mL DNase I for 30 min at 37° C., triturating with a 1000 μL pipette every 10 min to generate a single cell suspension. An equal volume of L-15 medium containing 20% ovomucoid protease inhibitor (Worthington Biochemical) is added, and the dissociated cells are passed through a 20 μm filter (PluriSelect) to remove large debris. The cells are pelleted at 350g for 5 min, washed with PBS, and then pelleted and resuspended in PBS containing 0.1% BSA. Finally, a 10 μL sample of the cell suspension is counted on a Luna FI automated counter using a Live/Dead assay (Thermo Fisher Scientific).

### Single-Cell Capture, Library Preparation and RNA-Seq

The cell suspension is diluted to a concentration of ˜1,000 cells per μL and immediately captured, lysed, and primed for reverse transcription (RT) using the high throughput, droplet microfluidics Gemcode platform from 10× Genomics. Each droplet on the Gemcode co-encapsulates a cell and a gel bead that is hybridized with oligo(dT) primers encoding a unique cell barcode and unique molecular identifiers (UMIs) in lysis buffer. The capture process takes 6.5 min, after which the transcriptomes captured on gel beads are immediately reverse transcribed to cDNA. Since all cDNA is pooled and PCR amplified, cell barcodes and UMIs facilitate demultiplexing of the originating cell and mRNA transcript after sequencing. RT, PCR amplification of cDNA, and preparation of a library from 3′ ends are conducted according to the manufacturer's published protocol. The libraries are sequenced on an Illumina NovaSeq 6000 instrument with 26 base pairs (bp) for the first read, 75 bp for the second read, and 8 bp for the index read1.

### Processing and QC of Single-Cell RNA-Seq Data

Reads are demultiplexed, aligned to the GRCm38 mm10 assembly reference genome (or latest available) with the sequence for the Atoh1 transgene appended, and filtered; and cell barcodes and UMIs are quantified using the 10X Genomics CellRanger pipeline with default parameters (software.10xgenomics.com/single-cell/overview/welcome). For each gene, UMI counts of all transcript isoforms are summed to obtain a digital measure of total gene expression. Droplets containing cells are selected from empty droplets based on ranked UMI complexity of the cell barcodes. This results in a digital expression matrix of genes by cells. All further filtering and downstream analysis of single-cell data described in subsequent sections is performed with Seurat, using default parameters unless specified. To limit the influence of low complexity cells and genes, cells with fewer than 100 expressed genes (i.e., transcript count >1), and genes with detectable expression in 10 or fewer cells are removed.

### Dimensionality Reduction, Clustering and Supporting Cell Classification

Full descriptions of Seurat's clustering procedure can be found at satijalab.org/seurat/. Briefly, after QC and filtering, gene expression for each cell is normalized by total transcript count, multiplied by a factor of 10,000, and log-transformed. To identify highly variable genes that account for cellular heterogeneity, genes are binned by average expression level and selected based on the z-score of their dispersion within each bin. For unbiased clustering of cells, Seurat uses a modularity-based method on shared nearest neighbor graphs, which are constructed from the Euclidean distances of reduced dimensionality space. Modularity optimization is then applied to cluster the cells, with the degree of clustering controlled by a user-defined resolution parameter. Principal component analysis (PCA) is used for dimensionality reduction and is calculated from z-scored residuals of the regressed gene expression matrix. PCA is first computed for the highly variable genes then projected onto the entire matrix.

### Comparison of Expression Levels in Supporting Cells

Clustering is first performed using the number of PCs accounting for 75% of the variance in the dataset, and a resolution of 1. Clusters expressing known supporting cell markers (such as Sox2, Jag1, Sall2, Lfng, and Kremen1) are selected. Once selected, the supporting cell-specific RNA-Seq profiles are then divided into groups according to vector/promoter they received, and expression levels are compared using Seurat's built-in plotting and significance testing functions. If a promoter drives Atoh1 transgene expression in supporting cells at level that is at least 0.25 log fold change higher than the long GFAP promoter, it is determined to be a high expression promoter. A high expression promoter can express Atoh1 transgene in supporting cells at a level that is at least 0.25, 0.5, 0.75, 1.0, 1.25, 1.5, 1.75, or 2.0 log fold change higher than the long GFAP promoter in this assay.

### Method for Identifying a Supporting Cell-Specific Promoter

In some embodiments, the promoter used in the compositions and methods described herein is a high expression, supporting cell-specific promoter. A promoter can be identified as a high expression promoter using the method described above. A promoter can be identified as a supporting cell-specific promoter using the following assay.

AAV encoding H2BGFP under the control of the promoter to be tested is injected into the left posterior canal of C56B1/6J mice (6-8-week-old) at a dose of 1 E10 gc/ear (1 μL total volume injected). Fourteen days later, mice are sacrificed and fixed with formalin via cardiac perfusion. Temporal bones are removed, decalcified in 8% EDTA for 3 days, embedded in paraffin, and sectioned on a microtome. Slides are stained with antibodies to GFP and haemotoxylin and imaged with a Leica Aperio digital slide scanner. The specificity of the promoter for supporting cells is determined based detection of GFP immunolabeling above background in supporting cells, hair cells, epithelial cells (e.g., nonsensory epithelium and roof epithelium), glial cells, and neurons.

A promoter is identified as a supporting cell-specific promoter if GFP immunolabeling above background is detected in at least 50% of supporting cells (e.g., at least 60, 70, 80, or 90% of supporting cells) and in less than 20% of hair cells (e.g., less than 15, 10, 5, or 1% of hair cells). In some embodiments, a supporting cell-specific promoter provides GFP immunolabeling above background in at least 90% of supporting cells and in less than 1% of hair cells. In some embodiments, GFP immunolabeling above background is also detected in less than 60% (e.g., less than 50%, 40%, 30%, 20%, 10%, or 5%) of other inner ear cell types (e.g., epithelial cells (e.g., nonsensory epithelium and roof epithelium), glial cells, and neurons). For reference, after injection of AAV8-short GFAP-H2BGFP, positive GFP immunolabeling can be detected in 100% of vestibular supporting cells, 0% of vestibular hair cells, and <50% of other inner ear cell types.

### Expression of Exogenous Nucleic Acids in Mammalian Cells

The compositions and methods described herein can be used to induce or increase the expression of Atoh1 in supporting cells (e.g., human cochlear and/or vestibular supporting cells) by administering a nucleic acid vector that contains a high expression promoter, such as a high expression-supporting cell-specific promoter (e.g., a GFAP promoter having the sequence of formula A-B-C, in which all or part of B is optionally absent, such as a GFAP promoter having the sequence of SEQ ID NO: 8) operably linked to a nucleic acid sequence that encodes Atoh1 (e.g., human Atoh1). A wide array of methods has been established for the delivery of proteins to mammalian cells and for the stable expression of genes encoding proteins in mammalian cells.

### Polynucleotides Encoding Proteins of Interest

One platform that can be used to achieve therapeutically effective intracellular concentrations of a protein of interest (e.g., Atoh1) in mammalian cells is via the stable expression of the gene encoding the protein of interest (e.g., by integration into the nuclear or mitochondrial genome of a mammalian cell or by episomal concatemer formation in the nucleus of a mammalian cell). The gene is a polynucleotide that encodes the primary amino acid sequence of the corresponding protein. In order to introduce an exogenous gene into a mammalian cell, the gene can be incorporated into a vector, Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

Proteins of interest can also be introduced into a mammalian cell by targeting a vector containing a gene encoding a protein of interest to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding a protein of interest by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith, et al., Mol. Sys. Biol., 3:73, online publication, the disclosure of which is incorporated herein by reference. The promoter used in the methods and compositions described herein is a high expression promoter, such as a high expression, supporting cell-specific promoter (e.g., a GFAP promoter having the sequence of formula A-B-C, in which all or part of B is optionally absent, such as a GFAP promoter having the sequence of SEQ ID NO: 8).

Once a polynucleotide encoding a protein of interest has been incorporated into a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, Calif.) and can be administered to a mammalian cell in order to promote gene expression according to established protocols.

Other DNA sequence elements that may be included in polynucleotides for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode a protein of interest (e.g., Atoh1) and additionally include a mammalian enhancer sequence. Many enhancer sequences are now known from mammalian genes, and examples include enhancers from the genes that encode mammalian globin, elastase, albumin, a-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription include the CMV enhancer and RSV enhancer. An enhancer may be spliced into a vector containing a polynucleotide encoding a protein of interest, for example, at a position 5′ or 3′ to this gene. In a preferred orientation, the enhancer is positioned at the 5′ side of the promoter, which in turn is located 5′ relative to the polynucleotide encoding a protein of interest.

The nucleic acid vectors described herein containing a high expression promoter, such as a high expression, supporting cell-specific promoter, operably linked to a polynucleotide encoding Atoh1 may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo. In some embodiments of the compositions and methods described herein, the WPRE has the sequence:

(SEQ ID NO: 16)
GATCCAATCAACCTCTGGATTACAAAATTTGTGAA
AGATTGACTGGTATTCTTAACTATGTTGCTCCTTT
TACGCTATGTGGATACGCTGCTTTAATGCCTTTGT
ATCATGCTATTGCTTCCCGTATGGCTTTCATTTTC
TCCTCCTTGTATAAATCCTGGTTGCTGTCTCTTTA
TGAGGAGTTGTGGCCCGTTGTCAGGCAACGTGGCG
TGGTGTGCACTGTGTTTGCTGACGCAACCCCCACT
GGTTGGGGCATTGCCACCACCTGTCAGCTCCTTTC
CGGGACTTTCGCTTTCCCCCTCCCTATTGCCACGG
CGGAACTCATCGCCGCCTGCCTTGCCCGCTGCTGG
ACAGGGGCTCGGCTGTTGGGCACTGACAATTCCGT
GGTGTTGTCGGGGAAATCATCGTCCTTTCCTTGGC
TGCTCGCCTGTGTTGCCACCTGGATTCTGCGCGGG
ACGTCCTTCTGCTACGTCCCTTCGGCCCTCAATCC
AGCGGACCTTCCTTCCCGCGGCCTGCTGCCGGCTC
TGCGGCCTCTTCCGCGTCTTCGA.

In other embodiments, the WPRE has the sequence:

(SEQ ID NO: 17)
AATCAACCTCTGGATTACAAAATTTGTGAMGATTG
ACTGGTATTCTTAACTATGTTGCTCCTTTTACGCT
ATGTGGATACGCTGCTTTAATGCCTTTGTATCATG
CTATTGCTTCCCGTATGGCTTTCATTTTCTCCTCC
TTGTATAAATCCTGGTTAGTTCTTGCCACGGCGGA
ACTCATCGCCGCCTGCCTTGCCCGCTGCTGGACAG
GGGCTCGGCTGTTGGGCACTGACAATTCCGTGGTG
TTTATTTGTGAAATTTGTGATGCTATTGCTTTATT
TGTAACCATCTAGCTTTATTTGTGAAATTTGTGAT
GCTATTGCTTTATTTGTAACCATTATAAGCTGCAA
TAAACAAGTTAACAACAACAATTGCATTCATTTTA
TGTTTCAGGTTCAGGGGGAGATGTGGGAGGTTTTT
TAAA

In some embodiments, the nucleic acid vectors containing a GFAP promoter described herein include a reporter sequence, which can be useful in verifying the expression of a gene operably linked to a GFAP promoter in VSCs. Reporter sequences that may be included in a nucleic acid vector described herein include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements that drive their expression, such as a GFAP promoter, the reporter sequences provide signals detectable by conventional means, including enzymatic, radiographic, colorimetric, fluorescence or other spectrographic assays, fluorescent activating cell sorting assays, and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for p-galactosidase activity. Where the transgene is green fluorescent protein or luciferase, the vector carrying the signal may be measured visually by color or light production in a luminometer.

### Methods for the Delivery of Exogenous Nucleic Acids to Target Cells

Techniques that can be used to introduce a transgene, such as a transgene (e.g., Atoh1) operably linked to a high expression promoter described herein (e.g., a high expression, supporting cell-specific promoter, such as a GFAP promoter having the sequence of formula A-B-C, in which all or part of B is optionally absent, such as a GFAP promoter having the sequence of SEQ ID NO: 8), into a target cell (e.g., a mammalian cell, e.g., a human supporting cell) are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) polyethyleneimine, and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for instance, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane to permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Vectors for Delivery of Exogenous Nucleic Acids to Target Cells

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are described in, e.g., Gellissen, Production of Recombinant Proteins: Novel Microbial and Eukaryotic Expression Systems (John Wiley & Sons, Marblehead, M A, 2006). Expression vectors for use in the compositions and methods described herein contain a high expression promoter (e.g., a high expression, supporting cell-specific promoter, such as a GFAP promoter having the sequence of formula A-B-C, in which all or part of B is optionally absent, such as a GFAP promoter having the sequence of SEQ ID NO: 8) operably linked to a polynucleotide sequence that encodes Atoh1, as well as, e.g., additional sequence elements used for the expression of these agents and/or the integration of these polynucleotide sequences into the genome of a mammalian cell. Vectors that can contain a high expression promoter operably linked to a transgene encoding Atoh1 include plasmids (e.g., circular DNA molecules that can autonomously replicate inside a cell), cosmids (e.g., pWE or sCos vectors), artificial chromosomes (e.g., a human artificial chromosome (HAC), a yeast artificial chromosome (YAC), a bacterial artificial chromosome (BAC), or a P1-derived artificial chromosome (PAC)), and viral vectors. Certain vectors that can be used for the expression of a protein of interest (e.g., Atoh1) include plasmids that contain regulatory sequences, such as enhancer regions, which direct gene transcription. Other useful vectors for expression of a protein of interest contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Viral Vectors for Nucleic Acid Delivery

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Nucleic Acid Delivery

In some embodiments, polynucleotides of the compositions and methods described herein are incorporated into rAAV vectors and/or virions in order to facilitate their introduction into a cell (e.g., a supporting cell). rAAV vectors useful in the compositions and methods described herein are recombinant nucleic acid constructs that include (1) a high expression promoter (e.g., a high expression, supporting cell-specific promoter, such as a GFAP promoter having the sequence of formula A-B-C, in which all or part of B is optionally absent, such as a GFAP promoter having the sequence of SEQ ID NO: 8), (2) a heterologous sequence to be expressed (e.g., a polynucleotide encoding Atoh1), and (3) viral sequences that facilitate stability and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part, but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The polynucleotides and vectors described herein (e.g., a high expression promoter operably linked to a transgene encoding Atoh1) can be incorporated into a rAAV virion in order to facilitate introduction of the polynucleotide or vector into a cell (e.g., a cochlear and/or vestibular supporting cell). The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, 11, rh10, rh39, rh43, rh74, Anc80, Anc80L65, DJ/8, DJ/9, 7m8, PHP.B, PHP.eB, and PHP.S. For targeting supporting cells, AAV1, AAV2, AAV2quad(Y-F), AAV3, AAV4, AAV5, AAV6, AAV7, AAV8, AAV9, Anc80, 7m8, PHP.B, PHP.eB, or PHP.S serotypes may be particularly useful. Serotypes evolved for transduction of the retina may also be used in the methods and compositions described herein. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV2quad(Y-F), AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

In some embodiments, the nucleic acid vector (e.g., an AAV vector) includes a high expression promoter described herein (e.g., a high expression, supporting cell-specific promoter described herein, such as the GFAP promoter of SEQ ID NO: 8) operably linked to a polynucleotide sequence encoding human Atoh1 (human ATOH1 protein=RefSeq Accession No. NP_005163 (SEQ ID NO: 10); mRNA sequence=RefSeq Accession No. NM_005172). In some embodiments, the high expression promoter is the high expression, supporting cell-specific GFAP promoter of SEQ ID NO: 8 (also represented by nucleotides 228-908 of SEQ ID NO: 15) and it is operably linked to a polynucleotide sequence encoding human Atoh1. In some embodiments, the polynucleotide sequence encoding human Atoh1 is SEQ ID NO: 11. In some embodiments, the polynucleotide sequence encoding human Atoh1 is nucleotides 925-1986 of SEQ ID NO: 15. In some embodiments, the polynucleotide sequence that encodes human Atoh1 is any polynucleotide sequence that, by redundancy of the genetic code, encodes SEQ ID NO: 10. The polynucleotide sequence that encodes human Atoh1 can be partially or fully codon-optimized for expression. In some embodiments, the vector includes, in 5′ to 3′ order, a first inverted terminal repeat; a GFAP promoter of SEQ ID NO: 8; a polynucleotide sequence encoding human Atoh1 operably linked to the GFAP promoter; a polyadenylation sequence; and a second inverted terminal repeat. In some embodiments, the nucleic acid vector includes, in 5′ to 3′ order, a first inverted terminal repeat; a GFAP promoter of SEQ ID NO: 8; a polynucleotide sequence encoding human Atoh1 operably linked to the GFAP promoter; a Woodchuck Posttranscriptional Regulatory Element (WPRE); a polyadenylation sequence; and a second inverted terminal repeat. In some embodiments, the WPRE has the sequence of SEQ ID NO: 16 or SEQ ID NO: 17. In some embodiments, the WPRE has the sequence of SEQ ID NO: 16. In some embodiments, the WPRE has the sequence of nucleotides 1997-2544 of SEQ ID NO: 15. In some embodiments, the polyadenylation sequence has the sequence of nucleotides 2557-2764 of SEQ ID NO: 15. In certain embodiments, the nucleic acid vector includes nucleotides 228-2764 of SEQ ID NO: 15, flanked by inverted terminal repeats. In some embodiments, the flanking inverted terminal repeats are AAV2 inverted terminal repeats. In some embodiments, the flanking inverted terminal repeats are any variant of AAV2 inverted terminal repeats that can be encapsidated by a plasmid that carries the AAV2 Rep gene. In particular embodiments, the nucleic acid vector includes nucleotides 228-2764 of SEQ ID NO: 15, flanked by inverted terminal repeats, in which the 5′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 1-130 of SEQ ID NO: 15; and in which the 3′ inverted terminal repeat has at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to nucleotides 2852-2981 of SEQ ID NO: 15. In some embodiments, the nucleic acid vector is a viral vector. In some embodiments, the viral vector is an AAV vector. In some embodiments, the AAV vector has an AAV8 capsid.

It should be understood by those of ordinary skill in the art that the creation of a viral vector of the invention typically requires the use of a plasmid of the invention together with additional plasmids that provide required elements for proper viral packaging and viability (e.g., for AAV, plasmids providing the appropriate AAV rep gene, cap gene and other genes (e.g., E2A and E4)). The combination of those plasmids in a producer cell line produces the viral vector. However, it will be understood by those of skill in the art, that for any given pair of inverted terminal repeat sequences in a transfer plasmid of the invention (e.g., SEQ ID NO: 14 or 15) that is used to create the viral vector, the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom. However, in some very specific embodiments, the viral vector of the invention comprises nucleotides 1-2981 of SEQ ID NO: 15.

### Pharmaceutical Compositions

The nucleic acid vectors described herein (e.g., vectors containing a high expression promoter, such as a high expression, supporting cell-specific promoter, e.g., a GFAP promoter having the sequence of formula A-B-C, in which all or part of B is optionally absent, such as a GFAP promoter having the sequence of SEQ ID NO: 8 operably linked to a polynucleotide encoding Atoh1) can be incorporated into a vehicle for administration into a patient, such as a human patient suffering from hearing loss and/or vestibular dysfunction. Pharmaceutical compositions containing vectors, such as viral vectors, that contain a high expression promoter operably linked to a polynucleotide encoding Atoh1 can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of nucleic acid vectors (e.g., viral vectors) containing high expression promoter (e.g., a high expression, supporting cell-specific promoter, such as a GFAP promoter having the sequence of formula A-B-C, in which all or part of B is optionally absent, such as a GFAP promoter having the sequence of SEQ ID NO: 8) operably linked to a polynucleotide encoding Atoh1 may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the middle or inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject having or at risk of developing sensorineural hearing loss and/or vestibular dysfunction by a variety of routes, such as local administration to the middle or inner ear (e.g., administration into the perilymph or endolymph, such as to or through the oval window, round window, or semicircular canal (e.g., the horizontal canal), or by transtympanic or intratympanic injection, e.g., administration to a supporting cell of the inner ear), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly).

Subjects that may be treated as described herein are subjects having or at risk of developing sensorineural hearing loss and/or vestibular dysfunction (e.g., subjects having or at risk of developing hearing loss, vestibular dysfunction, or both). The compositions and methods described herein can be used to treat subjects having or at risk of developing damage to cochlear hair cells (e.g., damage related to acoustic trauma, disease or infection, head trauma, ototoxic drugs, or aging), subjects having or at risk of developing damage to vestibular hair cells (e.g., damage related to disease or infection, head trauma, ototoxic drugs, or aging), subjects having or at risk of developing sensorineural hearing loss, deafness, or auditory neuropathy, subjects having or at risk of developing vestibular dysfunction (e.g., dizziness, vertigo, imbalance, bilateral vestibulopathy (bilateral vestibular hypofunction), oscillopsia, or a balance disorder), subjects having tinnitus (e.g., tinnitus alone, or tinnitus that is associated with sensorineural hearing loss or vestibular dysfunction), subjects having a genetic mutation associated with hearing loss and/or vestibular dysfunction, or subjects with a family history of hereditary hearing loss, deafness, auditory neuropathy, tinnitus, or vestibular dysfunction. In some embodiments, the disease associated with damage to or loss of hair cells (e.g., cochlear and/or vestibular hair cells) is an autoimmune disease or condition in which an autoimmune response contributes to hair cell damage or death. Autoimmune diseases linked to sensorineural hearing loss and vestibular dysfunction include autoimmune inner ear disease (AIED), polyarteritis nodosa (PAN), Cogan's syndrome, relapsing polychondritis, systemic lupus erythematosus (SLE), Wegener's granulomatosis, Sjögren's syndrome, and Behçet's disease. Some infectious conditions, such as Lyme disease and syphilis can also cause hearing loss and vestibular dysfunction (e.g., by triggering autoantibody production). Viral infections, such as rubella, cytomegalovirus (CMV), lymphocytic choriomeningitis virus (LCMV), HSV types 1 &2, West Nile virus (WNV), human immunodeficiency virus (HIV) varicella zoster virus (VZV), measles, and mumps, can also cause hearing loss and vestibular dysfunction. In some embodiments, the subject has or is at risk of developing hearing loss and/or vestibular dysfunction that is associated with or results from loss of hair cells (e.g., cochlear or vestibular hair cells). The methods described herein may include a step of screening a subject for one or more mutations in genes known to be associated with hearing loss and/or vestibular dysfunction prior to treatment with or administration of the compositions described herein. A subject can be screened for a genetic mutation using standard methods known to those of skill in the art (e.g., genetic testing). The methods described herein may also include a step of assessing hearing and/or vestibular function in a subject prior to treatment with or administration of the compositions described herein. Hearing can be assessed using standard tests, such as audiometry, auditory brainstem response (ABR), electrocochleography (ECOG), and otoacoustic emissions. Vestibular function may be assessed using standard tests, such as eye movement testing (e.g., electronystagmogram (ENG) or videonystagmogram (VNG)), tests of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini and Horak, Eur J Phys Rehabil Med, 46:239 (2010). These tests can also be used to assess hearing and/or vestibular function in a subject after treatment with or administration of the compositions described herein. The compositions and methods described herein may also be administered as a preventative treatment to patients at risk of developing hearing loss and/or vestibular dysfunction, e.g., patients who have a family history of hearing loss or vestibular dysfunction (e.g., inherited hearing loss or vestibular dysfunction), patients carrying a genetic mutation associated with hearing loss or vestibular dysfunction who do not yet exhibit hearing impairment or vestibular dysfunction, or patients exposed to risk factors for acquired hearing loss (e.g., acoustic trauma, disease or infection, head trauma, ototoxic drugs, or aging) or vestibular dysfunction (e.g., disease or infection, head trauma, ototoxic drugs, or aging). The compositions and methods described herein can also be used to treat a subject with idiopathic vestibular dysfunction.

The compositions and methods described herein can be used to induce or increase hair cell regeneration in a subject (e.g., cochlear and/or vestibular hair cell regeneration). Subjects that may benefit from compositions that induce or increase hair cell regeneration include subjects suffering from hearing loss or vestibular dysfunction as a result of loss of hair cells (e.g., loss of hair cells related to trauma (e.g., acoustic trauma or head trauma), disease or infection, ototoxic drugs, or aging), and subjects with abnormal hair cells (e.g., hair cells that do not function properly when compared to normal hair cells), damaged hair cells (e.g., hair cell damage related to trauma (e.g., acoustic trauma or head trauma), disease or infection, ototoxic drugs, or aging), or reduced hair cell numbers due to genetic mutations or congenital abnormalities. The compositions and methods described herein can also be used to promote or increase cochlear and/or vestibular hair cell maturation, which can lead to improved hearing and/or vestibular function, respectively. In some embodiments, the compositions and methods described herein promote or increase the maturation of regenerated cochlear and/or vestibular hair cells (e.g., promote or increase the maturation of cochlear and/or vestibular hair cells formed in response to expression of a composition described herein, such as a composition containing a high expression, supporting cell-specific promoter, such as a promoter having the sequence of SEQ ID NO: 8, operably linked to Atoh1, in supporting cells).

The compositions and methods described herein can also be used to prevent or reduce hearing loss and/or vestibular dysfunction caused by ototoxic drug-induced hair cell damage or death (e.g., cochlear hair cell and/or vestibular hair cell damage or death) in subjects who have been treated with ototoxic drugs, or who are currently undergoing or soon to begin treatment with ototoxic drugs. Ototoxic drugs are toxic to the cells of the inner ear, and can cause sensorineural hearing loss, vestibular dysfunction (e.g., vertigo, dizziness, imbalance, bilateral vestibulopathy (bilateral vestibular hypofunction), or oscillopsia), tinnitus, or a combination of these symptoms. Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. In some embodiments, the methods and compositions described herein can be used to treat bilateral vestibulopathy (bilateral vestibular hypofunction) or oscillopsia. Bilateral vestibulopathy (bilateral vestibular hypofunction) and oscillopsia can be induced by aminoglycosides (e.g., the methods and compositions described herein can be used to promote or increase hair cell regeneration in a subject having or at risk of developing aminoglycoside-induced bilateral vestibulopathy (bilateral vestibular hypofunction) or oscillopsia).

Treatment may include administration of a composition containing a nucleic acid vector (e.g., an AAV vector) described herein in various unit doses. Each unit dose will ordinarily contain a predetermined-quantity of the therapeutic composition. The quantity to be administered, and the particular route of administration and formulation, are within the skill of those in the clinical arts. A unit dose need not be administered as a single injection but may include continuous infusion over a set period of time. Dosing may be performed using a syringe pump to control infusion rate in order to minimize damage to the inner ear (e.g., the cochlea and/or vestibular system).

The compositions described herein are administered in an amount sufficient to improve hearing, improve vestibular function (e.g., improve balance or reduce dizziness or vertigo), reduce tinnitus, treat bilateral vestibulopathy (bilateral vestibular hypofunction), treat oscillopsia, treat a balance disorder, increase or induce Atoh1 expression in supporting cells, increase or induce hair cell regeneration (e.g., cochlear and/or vestibular hair cell regeneration), increase hair cell numbers, or increase hair cell maturation (e.g., maturation of regenerated hair cells). Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100%, 125%, 150%, 200% or more) compared to hearing measurements obtained prior to treatment. Vestibular function may be evaluated using standard tests for balance and vertigo (e.g., eye movement testing (e.g., ENG or VNG), posturography, VOR testing (e.g., head impulse testing (Halrmagyi-Curthoys testing, e.g., VHIT), or caloric reflex testing), rotary-chair testing, ECOG, VEMP, and specialized clinical balance tests) and may be improved by 5% or more (e.g., 5%, 10%, 15%, 20%, 30%, 40%, 50%, 60%, 70%, 80%, 90%, 100%, 125%, 150%, 200% or more) compared to measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss and/or vestibular dysfunction (e.g., in subjects who carry a genetic mutation associated with hearing loss or vestibular dysfunction, who have a family history of hearing loss or vestibular dysfunction (e.g., hereditary hearing loss or vestibular dysfunction), or who have been exposed to risk factors associated with hearing loss or vestibular dysfunction (e.g., ototoxic drugs, head trauma, disease or infection, or acoustic trauma) but do not exhibit hearing impairment or vestibular dysfunction (e.g., vertigo, dizziness, or imbalance), or in subjects exhibiting mild to moderate hearing loss or vestibular dysfunction). Atoh1 expression may be evaluated using immunohistochemistry, Western blot analysis, quantitative real-time PCR, or other methods known in the art for detection of protein or mRNA, and may be increased by 5% or more (e.g., 5%, 10%, 15%, 20%, 30%, 40%, 50%, 60%, 70%, 80%, 90%, 100%, 125%, 150%, 200% or more) compared to expression prior to administration of the compositions described herein. Hair cell regeneration or maturation may be evaluated indirectly based on hearing tests or tests of vestibular function, and may be increased by 5% or more (e.g., 5%, 10%, 15%, 20%, 30%, 40%, 50%, 60%, 70%, 80%, 90%, 100%, 125%, 150%, 200% or more) compared to hair cell regeneration or maturation prior to administration of the compositions described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the composition depending on the dose and route of administration used for treatment. Depending on the outcome of the evaluation, the patient may receive additional treatments.

### Kits

The compositions described herein can be provided in a kit for use in promoting hair cell regeneration (e.g., cochlear and/or vestibular hair cell regeneration) and treating hearing loss (e.g., sensorineural hearing loss) or vestibular dysfunction (e.g., dizziness, imbalance, vertigo, bilateral vestibulopathy (bilateral vestibular hypofunction), a balance disorder, or oscillopsia). The kit may include a nucleic acid vector containing a high expression promoter (e.g., a high expression, supporting cell-specific promoter, such as a GFAP promoter having the sequence of formula A-B-C, in which all or part of B is optionally absent, such as a GFAP promoter having the sequence of SEQ ID NO: 8) operably linked to a polynucleotide encoding Atoh1 or a pharmaceutical composition containing such a nucleic acid vector. The nucleic acid vectors may be packaged in an AAV virus capsid (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. AAV-Atoh1 Regenerates Utricular Hair Cells in a Dose-Dependent Manner

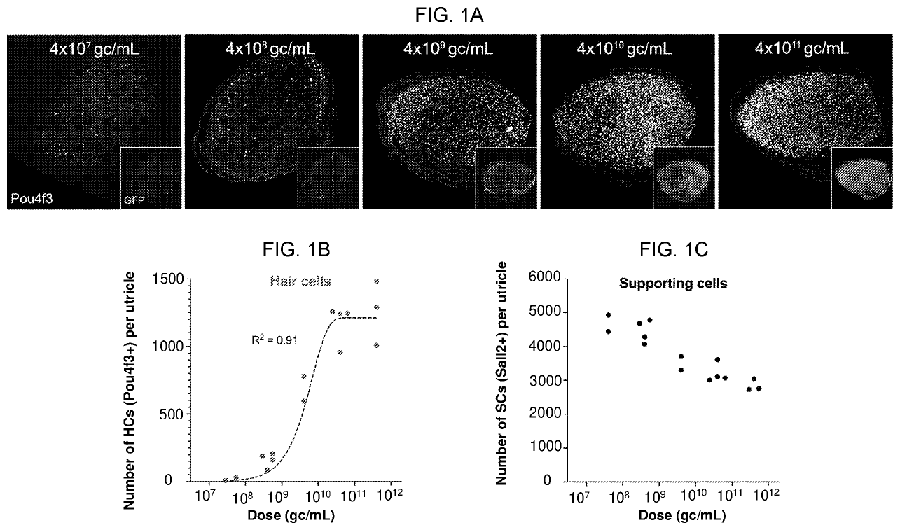

To evaluate the dose-dependence of hair cell regeneration, utricles were dissected from male C57Bl/6J mice (6-8-week-old) and cultured in 100 μL of base medium containing DMEM/F12 with 5% FBS and 2.5 μg/mL ciprofloxacin at 37° C. and 5% CO₂. Gentamicin (0.5 mg/mL) was added to the medium for 24 hours to kill hair cells, after which the gentamicin was washed out and replaced with 1 mL fresh medium for three days. AAV1-CMV-mouse Atoh1-2A-H2BGFP was then added to the culture medium at 4×10⁷ genome copies (gc)/mL, 4×10⁸ gc/mL, 4×10⁹ gc/mL, 4×10¹⁰ gc/mL, or 4×10¹¹ gc/mL. After three days of incubation, virus was washed out and utricles were cultured for an additional five days in 2 mL of fresh medium (12 days total culture time). At the end of the culture period, utricles were fixed with 4% PFA and immunohistochemistry was performed with antibodies to Pou4f3 and Sall2. Utricles were mounted and imaged on a Zeiss LSM 880, the number of hair cells (Pou4f3+ nuclei) and supporting cells (Sall2+ nuclei) per utricle were quantified with Imaris software. As shown in FIGS. 1A-1C, conversion of adult utricular supporting cells into hair cells via AAV-mediated Atoh1 overexpression was strongly dose-dependent. Confocal images of anti-Pou4f3 labeling in utricles treated with increasing doses of AAV1-CMV-mouse Atoh1-2A-H2BGFP showed a dose-dependent increase in hair cell regeneration (FIG. 1A). Insets show GFP expression throughout the utricle, which also increased with viral dose. The number of hair cells (Pou4f3+ nuclei) and supporting cells (Sall2+ nuclei) per utricle were graphed as a function of viral dose (FIGS. 1B-1 C). The increase in hair cells was fit by a one phase exponential association. Supporting cell numbers decreased with viral dose, indicating that hair cell regeneration was occurring via direct conversion of supporting cells into hair cells without an intervening mitosis.

### Example 2. Construction of an AAV Vector Containing a Short GFAP Promoter Operably Linked to a Polynucleotide Encoding Atoh1

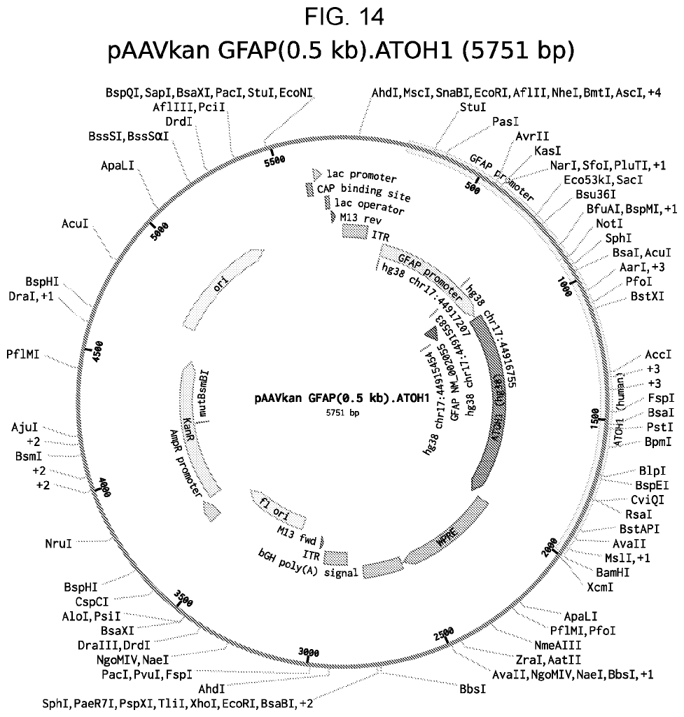

“AAV8-GFAP (SEQ ID NO: 8; “short GFAP” promoter)-hAtoh1-2A-H2BGFP” and “AAV8-short GFAP-hAtoh1-2A-H2BGFP” are used interchangeably and each refer to an AAV8 viral vector that contains nucleotides 228-3950 of SEQ ID NO: 14. “AAV8-short GFAP-hATOH1” refers to an AAV8 viral vector that contains nucleotides 228-2764 of SEQ ID NO: 15. The viral vector AAV8-short GFAP-hATOH1 was synthesized as follows. HEK293T cells (obtained from ATCC, Manassas, Va.) were seeded into cell culture-treated dishes (15 cm) and grown until they reached 70-80% confluence in the vessel. Plasmids were transfected into the 293T cells using conventional triple transfection methods: The transgene plasmid of SEQ ID NO: 15 (P712; FIG. 14) was combined with the plasmid pXR8 containing AAV2 rep/AAV8 cap (Addgene #112864) and the adenoviral helper plasmid pXX6-80 (X Xiao et al., J Virol 72(3), pp. 2224-32 (1998)) at a 1:1:1 molar ratio and 52.3 μg of that mixture was combined with PEIMax (Polysciences). A total of 52.3 μg of that plasmid mixture was delivered onto each 15 cm plate containing the cells. The cell culture medium and the cells were subsequently collected to extract and purify the AAV. AAV from the cells was released from cells through three cycles of freeze thaw, and the cell culture medium was collected to obtain secreted AAV. AAV from the cell culture medium was concentrated by adding PEG8000 to the solution, incubating at 4° C., and centrifuging to collect the AAV particles. All AAV was passed through iodixanol density gradient centrifugation to purify the AAV particles, and the buffer was exchanged to PBS with 0.01% pluronic F68 by passing the purified AAV and the buffer over a centrifugation column with a 100 kDa molecular weight cutoff. The other AAV viral vectors described herein were synthesized in a similar fashion using the appropriate transgene plasmid (which provides the promoter, the transgene(s), and other elements required for transgene expression).

### Example 3. Atoh1 Overexpression Level Correlates with the Efficiency of Supporting-Cell-to-Hair-Cell Conversion

To determine the relationship between the level of Atoh1 expression and conversion of adult utricular supporting cells into hair cells, utricles were transduced with AAVs in which the level of Atoh1 transgene expression was driven by promoters of three different strengths. Utricles were dissected from male C57Bl/6J mice (6-8-week-old) and cultured in 100 μL of base medium containing DMEM/F12 with 5% FBS and 2.5 μg/ml ciprofloxacin at 37° C. and 5% CO₂. Gentamicin (0.5 mg/mL) was added to the medium for 24 hours to kill hair cells, after which the gentamicin was washed out and replaced with 250 μL fresh medium containing one of the following AAVs at a dose of 1 E12 gc: AAV8-CMV-mouse Atoh1-2A-H2BGFP (very high expression), AAV8-GFAP (SEQ ID NO: 8; “short GFAP” promoter)-mouse Atoh1-2A-H2BGFP (high expression), AAV8-RLBP1-mouse Atoh1-2A-H2BGFP (low expression).

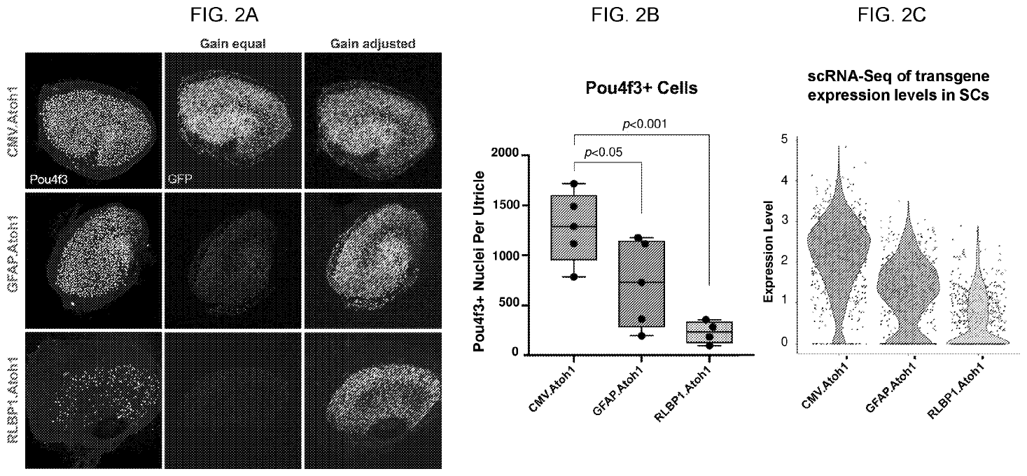

After one day of incubation, virus was washed out and utricles were cultured for an additional seven days in 2 mL of fresh medium (nine days total culture time). At the end of the culture period, some utricles were fixed with 4% PFA and immunohistochemistry was performed with antibodies to Pou4f3. Utricles were mounted and imaged on a Zeiss LSM 880, the number of hair cells (Pou4f3+ nuclei) per utricle was quantified with Imaris software. Alternatively, some utricles were dissociated and single cells were captured and prepared for single-cell RNA-Seq with a 10X Genomics Chromium system. Sequencing was performed on an Illumina NovaSeq, reads were aligned with CellRanger, and downstream analysis was performed with Seurat. At equal viral doses, promoters that induced higher levels of Atoh1 expression stimulated higher levels of hair cell regeneration (FIGS. 2A-2B). The difference in promoter activity was observed via the H2BGFP signal (FIG. 2A, middle panel, microscope acquisition setting equal across all conditions). Adjusting the microscope acquisition settings to match the GFP intensity level (FIG. 2A, right panel) revealed that viral transduction was comparable and widespread throughout the sensory epithelium for each virus, despite the differences in expression level. The difference in regeneration was quantified by measuring hair cell counts (Pou4f3+ nuclei) from each condition (FIG. 2B). Violin plots of single-cell RNA-Seq data were produced to show the levels of Atoh1 transgene expression in supporting cells from each condition (FIG. 2C). The expression analysis confirmed the gradient in promoter activity across the three viruses.

### Example 4. The Short GFAP Promoter Induced Higher Levels of Transgene Expression in U87 Cells than a GFAP Promoter Having the Sequence of SEQ ID NO: 9 (“Long GFAP” Promoter)

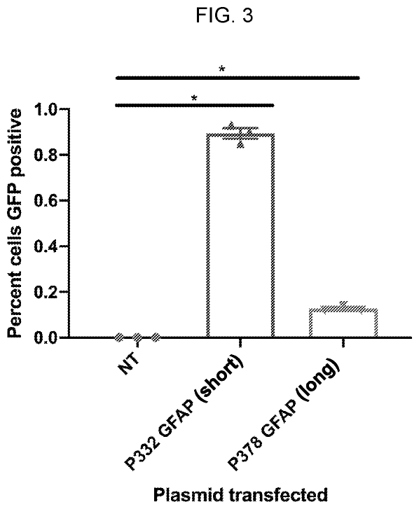

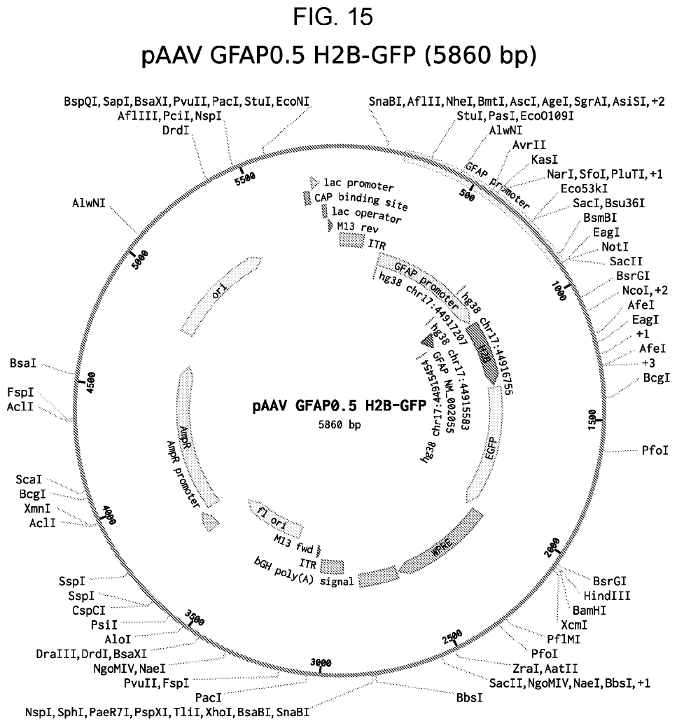

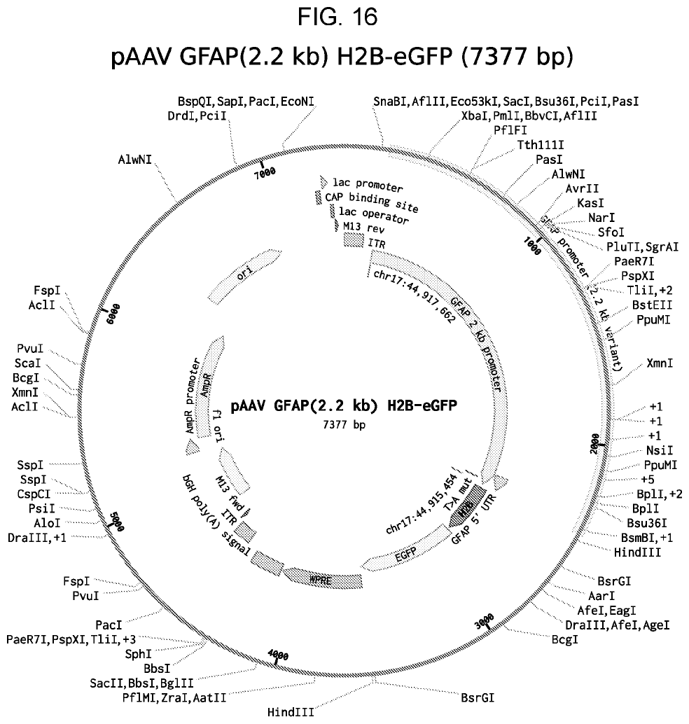

To evaluate transgene expression using two different GFAP promoters, U87 human glioblastoma cells were seeded at a density of 10,000 cells/well in a 96-well plate and cultured in DMEM+10% FBS+penicillin-streptomycin. One day after seeding, 100 ng of plasmid encoding either short GFAP-H2BGFP (plasmid P332; FIG. 15) or long GFAP-H2BGFP (plasmid P378; FIG. 16) was transfected into the cells using Lipofectamine 3000. Non-transfected cells (NT) were used as a control. Each of the three conditions was seeded and tested in triplicate. Two days after transfection, the cells were dissociated and the percentage of GFP+ cells for each condition was determined with flow cytometry on a Sony SH800 FACS machine. As shown in FIG. 3, a higher percentage of GFP+ cells were detected with the short GFAP promoter compared to the long GFAP promoter. Cells were transfected with equal amounts of plasmid, indicating that the increased detection rate was driven by higher levels of transgene expression, and, therefore, more cells with GFP levels surpassing the detection limit of the FACS machine.

### Example 5. The Short GFAP Promoter Induced Higher Levels of Transgene Expression in Utricle Explants than the Long GFAP Promoter

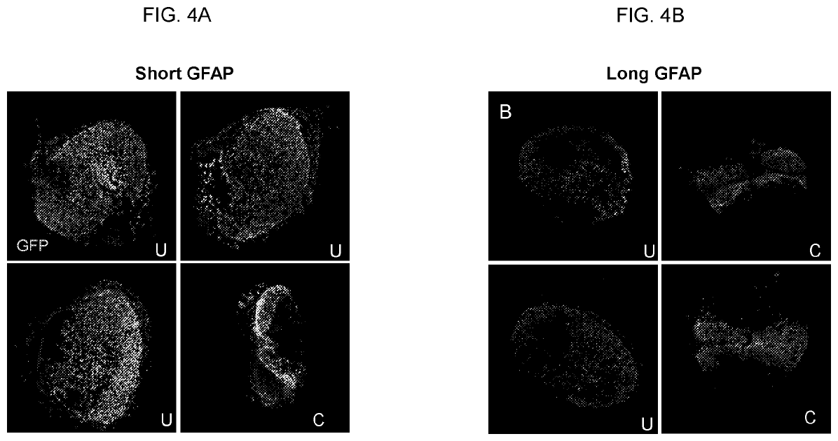

To evaluate transgene expression using two different GFAP promoters in utricles, utricles were dissected from male C57Bl/6J mice (11-week-old) and placed into 250 μL of culture medium containing DMEM/F12, 5% FBS, 2.5 μg/mL ciprofloxacin, and 2.5E11 gc of AAV8-short GFAP-H2BGFP or AAV8-long GFAP-H2BGFP at 37° C. and 5% CO₂. After one day of incubation, virus was washed out and utricles were cultured for an additional six days in 2 mL of fresh medium (seven days total culture time). At the end of the culture period, utricles were fixed with 4% PFA, mounted, and imaged on a Zeiss LSM 880. The intensity of H2BGFP in supporting cells was higher in the utricles (U) and cristae (C) treated with AAVs containing the short GFAP promoter (FIG. 4A) compared to the utricles (U) and cristae (C) treated with AAVs containing the long GFAP promoter (FIG. 4B). Since AAVs were transduced at equal doses, these data indicate that the short GFAP promoter induced higher levels of expression in supporting cells compared to the long GFAP promoter.

### Example 6. The Short GFAP Promoter is Active in Mouse Vestibular Supporting Cells In Vivo

To evaluate the activity of the short GFAP promoter in vivo, AAV8-short GFAP-H2BGFP was injected into the left posterior canal of C56B1/6J mice (6-8-week-old) at a dose of 1.51 E10 gc/ear (1 μL total volume injected). Fourteen days later, mice were sacrificed and fixed with formalin via cardiac perfusion. Temporal bones were removed, decalcified in EDTA, embedded in paraffin, and sectioned on a microtome. Slides were stained with chromogenic antibodies to GFP and haemotoxylin. Sections were imaged with a Leica Aperio digital slide scanner.

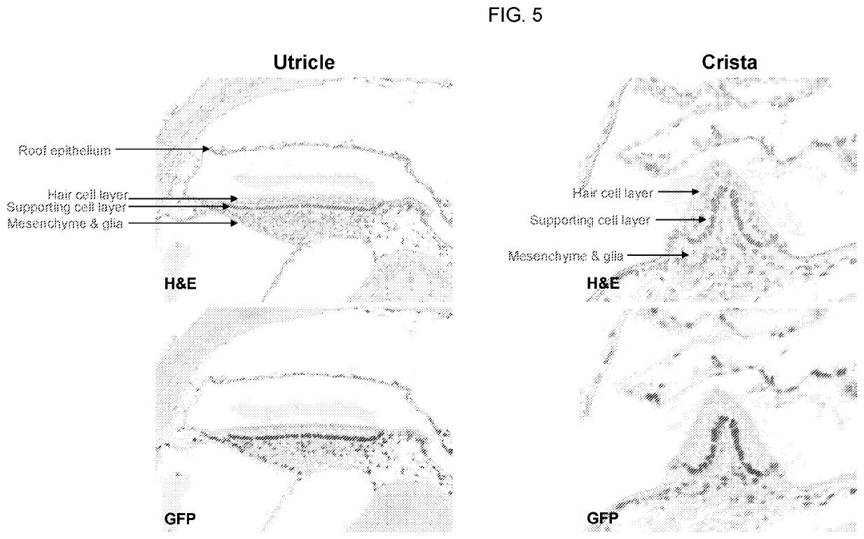

Intense nuclear GFP labeling was detected in all supporting cells in cross-sections of the utricle (FIG. 5, left) and crista (FIG. 5, right), but not in hair cells.

### Example 7. AAV8-Short GFAP-Atoh1 Robustly Regenerates Vestibular Hair Cells In Vivo

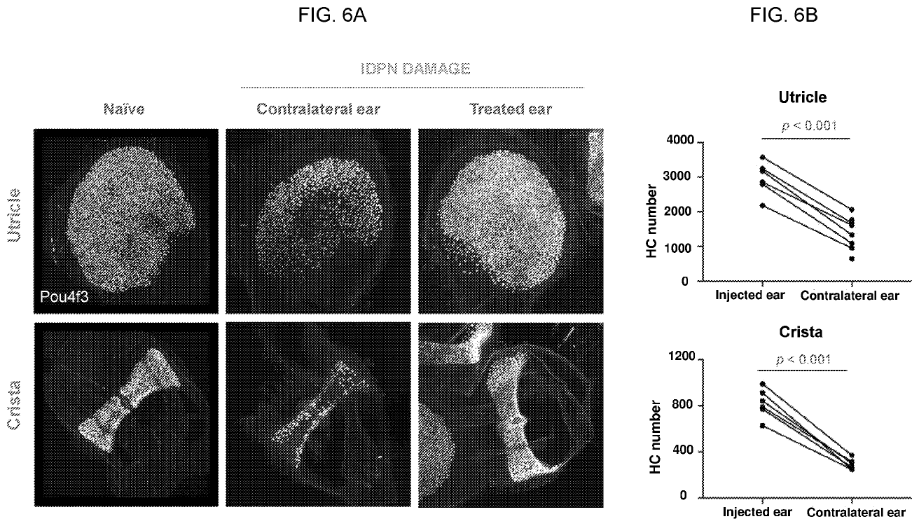

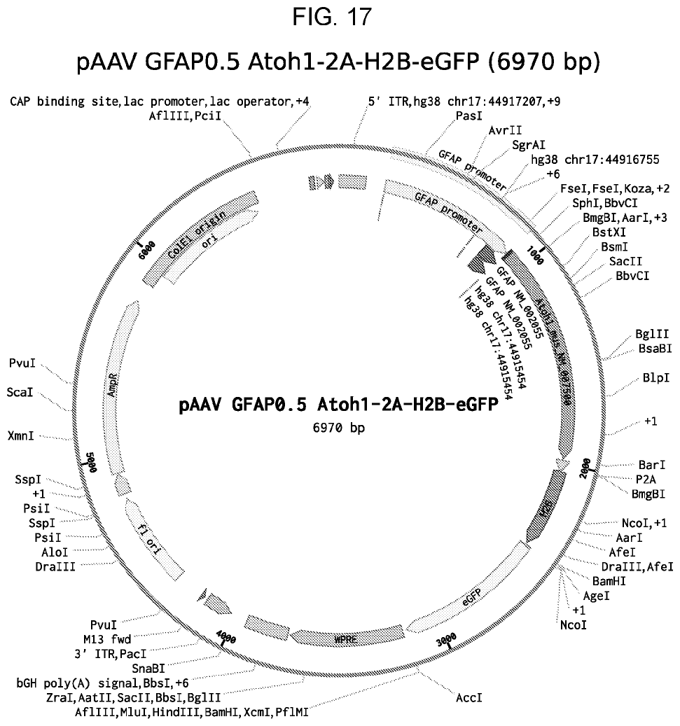

To evaluate the effect of an AAV8-short GFAP-mouse Atoh1-containing viral vector (synthesized using plasmid P319 (FIG. 17) as the transgene plasmid) on hair cell regeneration in vivo, a single I.P. injection of 5 g/kg IDPN was delivered to 8-9-week-old CD-1 mice (n=6). Fifteen to seventeen days later, 1 μL of AAV8-short GFAP-mouse Atoh1-2A-H2BGFP at a dose of 7.2E9 vg was delivered to the posterior semicircular canal (left ear only). Mice were allowed to survive for 13-14 days after virus delivery and then sacrificed and fixed with formalin via cardiac perfusion. Vestibular organs were microdissected and processed for immunohistochemistry. Organs were dissected from the ear of a naïve mouse not treated with IDPN or AAV (left), the contralateral ear of an IDPN-damaged mouse not treated with virus (middle), or the AAV-treated ear from the same IDPN-damaged mouse. FIG. 6A shows confocal images of utricles (top) and cristae (bottom) immunolabeled with antibodies to Pou4f3. IDPN caused a substantial decrease in hair cell numbers; however, treating with AAV8-short GFAP-mouse Atoh1 after IDPN damage robustly regenerated hair cells in both the utricle and cristae (FIG. 6A). FIG. 6B shows the quantification of hair cell numbers (based on Pou4f3 labeling) in utricles and cristae treated with AAV8-short GFAP-mouse Atoh1-2A-H2BGFP after IDPN damage compared to hair cells numbers in contralateral ears that were not treated with AAV; p<0.001, paired t-test.

### Example 8. Stereocilia Bundle Density is Increased in Regenerated Utricles

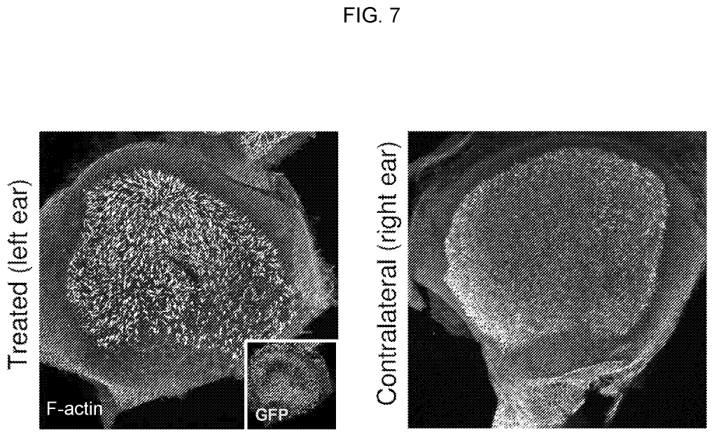

To evaluate the effect of AAV8-short GFAP-Atoh1 on stereocilia bundle density, a single I.P. injection of 5 g/kg IDPN was delivered to 8-week-old CD-1 mice (n=12). Fourteen to twenty-two days later, 1 μL of AAV8-short GFAP-human Atoh1-2A-H2BGFP at a dose of 2.5E10 vg was delivered to the posterior semicircular canal (left ear only). Mice were allowed to survive for 30 days after virus delivery and then sacrificed and fixed with formalin via cardiac perfusion. Vestibular organs were microdissected and processed for immunohistochemistry. Confocal imaging was used to observe fluorescent phalloidin labeling of F-actin in a utricle from an IDPN-damaged mouse that received AAV8-short GFAP-human Atoh1-2A-H2BGFP in its left ear (FIG. 7, left). A higher density of stereocilia bundles could be seen in this utricle compared to the utricle from the contralateral ear (FIG. 7, right) that did not receive virus. The inset image in the left panel shows GFP expression, confirming successful delivery of the virus.

### Example 9. Nerve Fiber and Synapse Density are Increased in Regenerated Utricles

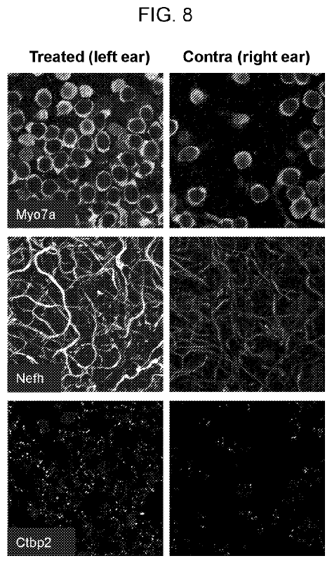

To evaluate the effect of an AAV8-short GFAP-human Atoh1-containing viral vector on nerve fiber and synapse density, a single I.P. injection of 5 g/kg IDPN was delivered to 8-week-old CD-1 mice (n=12). Fourteen to twenty-two days later, 1 μL of AAV8-short GFAP-human ATOH1-2A-H2BGFP at a dose of 2.5E10 vg was delivered to the posterior semicircular canal (left ear only). Mice were allowed to survive for 14 days after virus delivery and then sacrificed and fixed with formalin via cardiac perfusion. Vestibular organs were microdissected and processed for immunohistochemistry. Confocal images show immunostaining for Myo7a (hair cells), Nefh (nerve fibers), and Ctbp2 (ribbon synapses) in a utricle from an IDPN-damaged mouse that received AAV8-short GFAP-human Atoh1 in its left ear (FIG. 8, left). A higher density of nerve fibers and ribbon synapses was observed in this utricle compared to the utricle from the contralateral ear (FIG. 8, right) that did not receive virus.

### Example 10. Silencing Atoh1 Transgene Expression in New Hair Cells Via a Supporting-Cell-Specific Promoter Drives Further Maturation

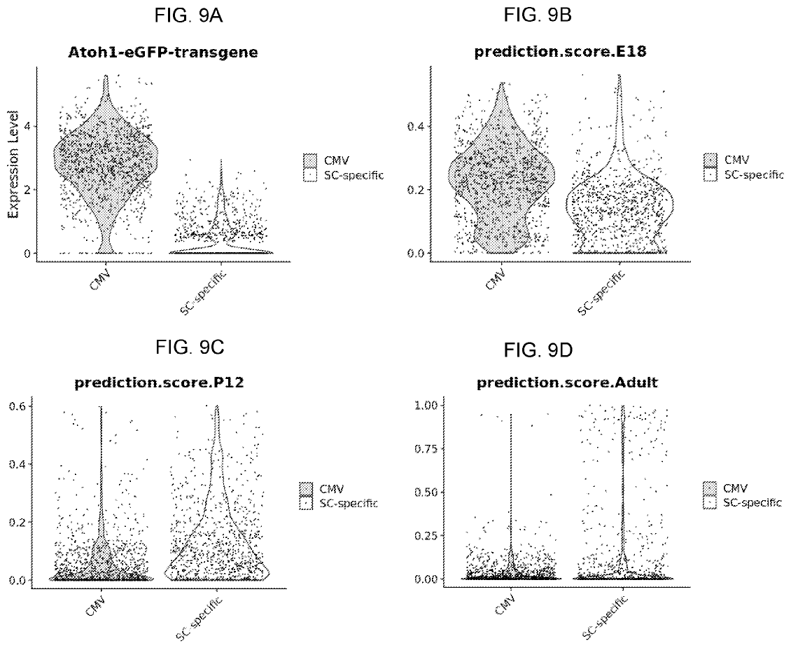

To evaluate the effect of promoter specificity on hair cell maturation, utricles were dissected from male C57Bl/6J mice (6-8-week-old) and cultured in 100 μL of base medium containing DMEM/F12 with 5% FBS and 2.5 μg/ml ciprofloxacin at 37° C. and 5% CO₂. Gentamicin (0.5 mg/mL) was added to the medium for 24 hours to kill hair cells, after which the gentamicin was washed out and replaced with 250 μL fresh medium containing one of the following AAVs at a dose of 1 E12 gc: AAV8-CMV-mouse Atoh1-2A-H2BGFP (CMV promoter group), AAV8-short GFAP-mouse Atoh1-2A-H2BGFP (SC-specific promoter group), AAV8-RLBP1-mouse Atoh1-2A-H2BGFP (SC-specific promoter group). After one day of incubation, virus was washed out and utricles were cultured for an additional 3, 8, or 16 days in 2 mL of fresh medium. At the end of the culture period, utricles were dissociated and single cells were captured and prepared for single-cell RNA-Seq with a 10X Genomics Chromium system. Sequencing was performed on an Illumina NovaSeq, reads were aligned with CellRanger, and downstream analysis was performed with Seurat. Prediction scores were generated in Seurat by comparing to databases of utricle hair cell single-cell RNA-Seq profiles that were generated from embryonic day 18 (E18), postnatal day 12 (P12), and adult mice. FIGS. 9A-9D are violin plots showing Atoh1 transgene expression and maturity prediction scores for regenerated hair cells in adult utricle explants treated with AAVs expressing mouse Atoh1 under the control of a ubiquitous CMV promoter or supporting-cell (SC)-specific promoters (short GFAP or RLBP1). The Atoh1 transgene was expressed at low or undetectable levels in regenerated hair cells in the SC-specific promoter group (FIG. 9A), whereas it was expressed at high levels in almost all hair cells from the CMV group. These results demonstrate that the Atoh1-transgene naturally downregulates in regenerated hair cells when it is driven by a SC-specific promoter. More of the single-cell RNA-Seq profiles from the SC-specific promoter group correlated strongly with P12 (FIG. 9C) and adult hair cells (FIG. 9D) than those from the CMV group. Conversely, more of the single-cell RNA-Seq profiles from the CMV group correlated strongly with E18 hair cells (FIG. 9B) than those from the SC-specific promoter group. Thus, natural silencing of the Atoh1 transgene with a SC-specific promoter drives maturation of regenerated hair cells.

### Example 11. “Low” and “High” Levels of Atoh1 Expression in Supporting Cells Generate Two Distinct Populations of Hair Cells

To evaluate the effect of Atoh1 expression levels in supporting cells on hair cell development, utricles were dissected from male C57Bl/6J mice (6-8-week-old) and cultured in 100 μL of base medium containing DMEM/F12 with 5% FBS and 2.5 μg/mL ciprofloxacin at 37° C. and 5% CO₂. Gentamicin (0.5 mg/mL) was added to the medium for 24 hours to kill hair cells, after which the gentamicin was washed out and replaced with 250 μL fresh medium containing one of the following AAVs at a dose of 1 E12 gc: AAV8-CMV-mouse Atoh1-2A-H2BGFP, AAV8-short GFAP-mouse Atoh1-2A-H2BGFP, or AAV8-RLBP1-mouse Atoh1-2A-H2BGFP. After one day of incubation, virus was washed out and utricles were cultured for an additional 3, 8, or 16 days in 2 mL of fresh medium. At the end of the culture period, utricles were dissociated and single cells were captured and prepared for single-cell RNA-Seq with a 10X Genomics Chromium system. Sequencing was performed on an Illumina NovaSeq, reads were aligned with CellRanger, and downstream analysis was performed with Seurat. Prediction scores were generated in Seurat by comparing to databases of utricle hair cell single-cell RNA-Seq profiles that were generated from E18 and P12 mice.

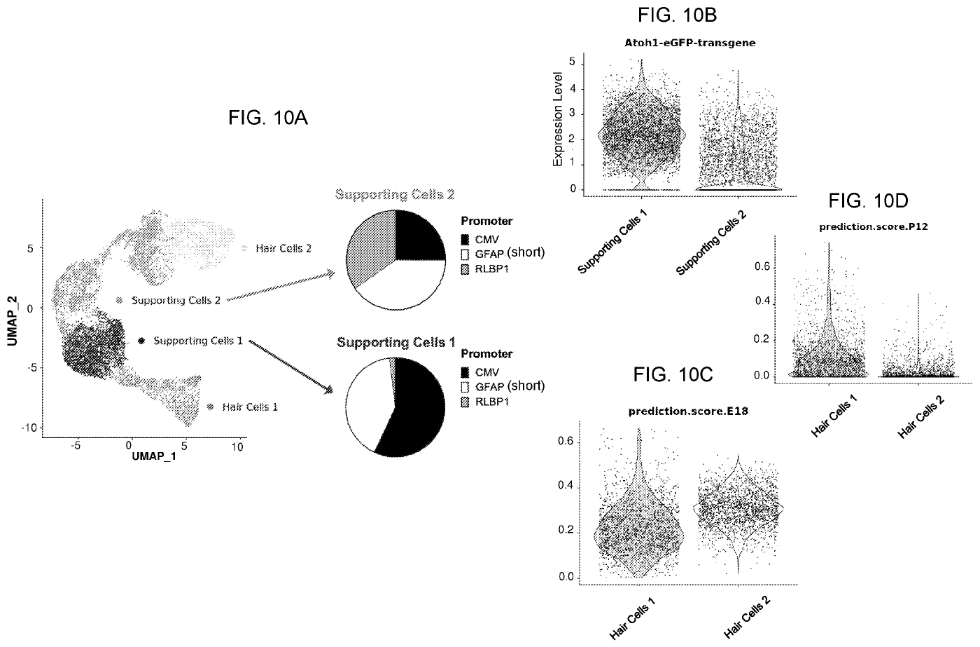

FIG. 10A shows a UMAP plot of single-cell RNA-Seq expression profiles generated from supporting cells and regenerated hair cells that were isolated from adult mouse utricle explants treated with AAV8-CMV-mouse Atoh1-2A-H2BGFP, AAV8-short GFAP-mouse Atoh1-2A-H2BGFP, or AAV8-RLBP1-mouse Atoh1-2A-H2BGFP. The supporting cells separated into two distinct clusters (labeled as Supporting Cells 1 and Supporting Cells 2) from which two clusters of regenerated hair cells appeared to originate (FIG. 10A, left). The supporting cells in cluster 1 were made up almost entirely of cells from samples treated with the short GFAP and CMV viruses, whereas almost all the supporting cells from the samples treated with RLBP1 virus fell in cluster 2 (FIG. 10A, right). Violin plots were produced to show Atoh1 transgene expression in the two supporting cell groups and demonstrated that cluster 1 had substantially higher levels of transgene expression compared to cluster 2 (FIG. 10B). Since both cluster 1 and 2 were made up of large numbers of cells from both the CMV and short GFAP virus conditions, the separation of the clusters was driven more by the difference in Atoh1 expression level as opposed to treatment type. RLBP1 is the weakest of the three promoters, and almost no cells from this treatment group reached high enough levels of Atoh1 expression to fall into cluster 1. Violin plots were also produced to show maturity prediction scores for regenerated hair cells from the two hair cell clusters (FIGS. 10C-10D). More of the single-cell RNA-Seq profiles from hair cell cluster 1 (hair cells generated from supporting cells with high levels of Atoh1 expression) correlated strongly with P12 hair cells than those from cluster 2 (FIG. 10D). Conversely, more of the single-cell RNA-Seq profiles from cluster 2 (hair cells generated from supporting cells with low levels of Atoh1 expression) correlated strongly with E18 hair cells than those from cluster 1 (FIG. 10C). Thus, higher levels of Atoh1 expression in supporting cells appeared to generate more mature hair cells than lower levels of Atoh1 expression. Weak promoters like RLBP1 were not able to drive sufficiently high levels of Atoh1 to generate many mature hair cells at the AAV doses used in this experiment.

### Example 12. AAV8-Short GFAP-hATOH1 Robustly Regenerates Vestibular Hair Cells In Vivo

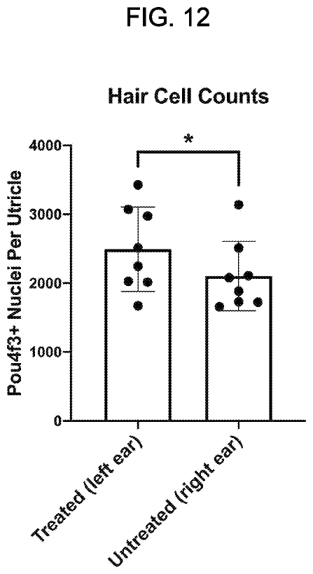

To evaluate the effect of AAV8-short GFAP-hATOH1 (human ATOH1 transgene with no GFP tag) on hair cell regeneration in vivo, a single I.P. injection of 4 g/kg IDPN was delivered to 8-9-week-old C57BL/6 mice (n=8). Fourteen days later, 1 μL of AAV8-short GFAP-hATOH1 at a dose of 1 E10 vg/ear was delivered to the posterior semicircular canal (left ear only). Mice were allowed to survive for 14 days after virus delivery and then sacrificed and fixed with formalin via cardiac perfusion. Utricles were microdissected and processed for immunohistochemistry. Hair cell nuclei were immunolabeled with antibodies raised against Pou4f3, and then utricles were mounted on glass slides and imaged with a Zeiss LSM800 confocal microscope. The number of hair cell nuclei in treated (left) ears and untreated (right) ears were quantified in the three-dimensional confocal z-stacks using Imaris software. As shown in FIG. 12, the number of hair cells in utricles treated with AAV8-short GFAP-hATOH1 after IDPN damage was significantly greater than the number of hair cells in contralateral ears that were not treated with AAV; p<0.05, paired t-test.

### Example 13. Administration of a Composition Containing a Nucleic Acid Vector Containing a High Expression, Supporting Cell-Specific Promoter Operably Linked to a Polynucleotide Encoding Atoh1 to a Subject with Vestibular Dysfunction

According to the methods disclosed herein, a physician of skill in the art can treat a patient, such as a human patient, with vestibular dysfunction (e.g., bilateral vestibulopathy) so as to improve or restore vestibular function (e.g., improve balance or reduce falls). To this end, a physician of skill in the art can administer to the human patient a composition containing an AAV vector (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S) containing a high expression, supporting cell specific promoter (e.g., a GFAP promoter having the sequence of SEQ ID NO: 8) operably linked to a polynucleotide encoding Atoh1 (e.g., human Atoh1). The composition containing the AAV vector may be administered to the patient, for example, by local administration to the inner ear (e.g., injection into a semicircular canal, such as the horizontal canal), to treat vestibular dysfunction.

Following administration of the composition to a patient, a practitioner of skill in the art can monitor the expression of the therapeutic protein encoded by the transgene, and the patient's improvement in response to the therapy, by a variety of methods. For example, a physician can monitor the patient's vestibular function by performing standard tests such as electronystagmography, video nystagmography, rotation tests, tests of the VOR, vestibular evoked myogenic potential, or computerized dynamic posturography. A finding that the patient exhibits improved vestibular function in one or more of the tests following administration of the composition compared to test results obtained prior to administration of the composition indicates that the patient is responding favorably to the treatment. Subsequent doses can be determined and administered as needed.

### Example 14. Administration of a Composition Containing a Nucleic Acid Vector Containing a High Expression, Supporting Cell-Specific Promoter Operably Linked to a Polynucleotide Encoding Atoh1 to a Subject with Sensorineural Hearing Loss

According to the methods disclosed herein, a physician of skill in the art can treat a patient, such as a human patient, with hearing loss (e.g., sensorineural hearing loss) so as to improve or restore hearing. To this end, a physician of skill in the art can administer to the human patient a composition containing an AAV vector (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S) containing a high expression, supporting cell specific promoter (e.g., a GFAP promoter having the sequence of SEQ ID NO: 8) operably linked to a polynucleotide encoding Atoh1 (e.g., human Atoh1). The composition containing the AAV vector may be administered to the patient, for example, by local administration to the inner ear (e.g., injection into the perilymph or to or through the round window membrane), to treat sensorineural hearing loss.

Following administration of the composition to a patient, a practitioner of skill in the art can monitor the patient's improvement in response to the therapy by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A nucleic acid vector comprising a high expression supporting cell-specific promoter operably linked to a polynucleotide encoding atonal BHLH transcription factor 1 (Atoh1).

2. The nucleic acid vector of claim 1, wherein the high expression supporting cell-specific promoter is a GFAP promoter having the sequence of formula A-B-C, wherein A has the sequence of SEQ ID NO: 1, B has the sequence of SEQ ID NO: 2, and C has the sequence of SEQ ID NO: 3, wherein all or part of B is optionally absent.

3. The nucleic acid vector of claim 2, wherein nucleotides 1-254 of B (SEQ ID NO: 4) are present.

4. The nucleic acid vector of claim 2 or 3, wherein nucleotides 230-483 of B (SEQ ID NO: 5) are present.

5. The nucleic acid vector of any one of claims 2-4, wherein nucleotides 459-711 of B (SEQ ID NO: 6) are present.

6. The nucleic acid vector of any one of claims 2-5, wherein nucleotides 687-917 of B (SEQ ID NO: 7) are present.

7. The nucleic acid vector of any one of claims 2-6, wherein all of B is present.

8. The nucleic acid vector of claim 1 or 2, wherein the high expression supporting cell-specific promoter has the sequence of SEQ ID NO: 8.

9. The nucleic acid vector of any one of claims 1-8, wherein the polynucleotide encodes an Atoh1 polypeptide having the sequence of SEQ ID NO: 10.

10. The nucleic acid vector of any one of claims 1-9, wherein the nucleic acid vector is a viral vector, plasmid, cosmid, or artificial chromosome.

11. The nucleic acid vector of claim 10, wherein the nucleic acid vector is a viral vector selected from the group consisting of an adeno-associated virus (AAV), an adenovirus, and a lentivirus.

12. The nucleic acid vector of claim 11, wherein the viral vector is an AAV vector.

13. The nucleic acid vector of claim 12, wherein the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S capsid.

14. A composition comprising the nucleic acid vector of any one of claims 1-13.

15. A cell comprising the nucleic acid vector of any one of claims 1-13.

16. The cell of claim 15, wherein the cell is a mammalian supporting cell.

17. The cell of claim 16, wherein the mammalian supporting cell is a human supporting cell.

18. The cell of claim 16 or 17, wherein the supporting cell is a VSC or a cochlear supporting cell.

19. A method of expressing Atoh1 in a mammalian supporting cell, the method comprising contacting the supporting cell with the nucleic acid vector of any one of claims 1-13 or the composition of claim 14.

20. The method of claim 19, wherein the mammalian cell is a human supporting cell.

21. The method of claim 19 or 20, wherein the mammalian supporting cell is a VSC or a cochlear supporting cell.

22. A method of inducing or increasing hair cell regeneration in a human subject in need thereof, comprising administering to the subject an effective amount of a nucleic acid vector encoding a high expression promoter operably linked to a polynucleotide encoding Atoh1.

23. A method of inducing or increasing hair cell maturation in a human subject in need thereof, the method comprising administering to the subject an effective amount of a nucleic acid vector encoding a high expression promoter operably linked to a polynucleotide encoding Atoh1.

24. The method of claim 22 or 23, wherein the hair cell is a vestibular hair cell.

25. The method of claim 24, wherein the vestibular hair cell is a Type II vestibular hair cell.

26. The method of claim 22 or 23, wherein the hair cell is a cochlear hair cell.

27. The method of claim 26, wherein the cochlear hair cell is an inner hair cell.

28. The method of claim 26, wherein the cochlear hair cell is an outer hair cell.

29. The method of any one of claims 22-25, wherein the subject has or is at risk of developing vestibular dysfunction.

30. The method of any one of claims 22, 23, and 26-28, wherein the subject has or is at risk of developing hearing loss.

31. A method of treating a human subject having or at risk of developing vestibular dysfunction, comprising administering to the subject an effective amount of a nucleic acid vector encoding a high expression promoter operably linked to a polynucleotide encoding Atoh1.

32. The method of claim 29 or 31, wherein the vestibular dysfunction comprises vertigo, dizziness, imbalance, bilateral vestibulopathy, oscillopsia, or a balance disorder.

33. The method of any one of claims 29, 31 and 32, wherein the vestibular dysfunction is age-related vestibular dysfunction, head trauma-related vestibular dysfunction, disease or infection-related vestibular dysfunction, or ototoxic drug-induced vestibular dysfunction.

34. The method of any one of claims 29, 31 and 32, wherein the vestibular dysfunction is associated with a genetic mutation.

35. A method of treating a human subject having or at risk of developing bilateral vestibulopathy, the method comprising administering to the subject an effective amount of a nucleic acid vector encoding a high expression promoter operably linked to a polynucleotide encoding Atoh1.

36. The method of claim 35, wherein the bilateral vestibulopathy is ototoxic drug-induced bilateral vestibulopathy.

37. A method of treating a human subject having or at risk of developing oscillopsia, the method comprising administering to the subject an effective amount of a nucleic acid vector encoding a high expression promoter operably linked to a polynucleotide encoding Atoh1.

38. The method of claim 37, wherein the oscillopsia is ototoxic drug-induced oscillopsia.

39. A method of treating a human subject having or at risk of developing a balance disorder, the method comprising administering to the subject an effective amount of a nucleic acid vector encoding a high expression promoter operably linked to a polynucleotide encoding Atoh1.

40. A method of treating a human subject having or at risk of developing hearing loss, the method comprising administering to the subject an effective amount of a nucleic acid vector encoding a high expression promoter operably linked to a polynucleotide encoding Atoh1.

41. The method of claim 30 or 40, wherein the hearing loss is genetic hearing loss.

42. The method of claim 41, wherein the genetic hearing loss is autosomal dominant hearing loss, autosomal recessive hearing loss, or X-linked hearing loss.

43. The method of claim 30 or 40, wherein the hearing loss is acquired hearing loss.

44. The method of claim 43, wherein the acquired hearing loss is noise-induced hearing loss, age-related hearing loss, disease or infection-related hearing loss, head trauma-related hearing loss, or ototoxic drug-induced hearing loss.

45. The method of claim 33, 36, 38, or 44, wherein the ototoxic drug is an aminoglycoside, an antineoplastic drug, ethacrynic acid, furosemide, a salicylate, or quinine.

46. A method of treating a human subject having or at risk of developing tinnitus, comprising administering to the subject an effective amount of a nucleic acid vector encoding a high expression promoter operably linked to a polynucleotide encoding Atoh1.

47. The method of any one of claims 22-25, 29, 31-39, and 45, wherein the method further comprises evaluating the vestibular function of the subject prior to administering the nucleic acid vector or composition.

48. The method of any one of claims 22-25, 29, 31-39, 45, and 47, wherein the method further comprises evaluating the vestibular function of the subject after administering the nucleic acid vector.

49. The method of any one of claims 22, 23, 26-28, and 40-46, wherein the method further comprises evaluating the hearing of the subject prior to administering the nucleic acid vector.

50. The method of any one of claims 22, 23, 26-28, 40-46, and 49, wherein the method further comprises evaluating the hearing of the subject after administering the nucleic acid vector.

51. The method of any one of claims 22-50, wherein the nucleic acid vector is locally administered.

52. The method of claim 51, wherein the nucleic acid vector is administered to the inner ear.

53. The method of claim 51, wherein the nucleic acid vector is administered to the middle ear.

54. The method of claim 51, wherein the nucleic acid vector is administered to a semicircular canal.

55. The method of claim 51, wherein the nucleic acid vector is administered transtympanically or intratympanically.

56. The method of claim 51, wherein the nucleic acid vector is administered into the perilymph.

57. The method of claim 51, wherein the nucleic acid vector is administered into the endolymph.

58. The method of claim 51, wherein the nucleic acid vector is administered to or through the oval window.

59. The method of claim 51, wherein the nucleic acid vector is administered to or through the round window.

60. The method of any one of claims 22-59, wherein the nucleic acid vector is the nucleic acid vector of any one of claims 1-13.

61. The method of any one of claims 22-60, wherein the nucleic acid vector is administered in an amount sufficient to prevent or reduce vestibular dysfunction, delay the development of vestibular dysfunction, slow the progression of vestibular dysfunction, improve vestibular function, prevent or reduce hearing loss, prevent or reduce tinnitus, delay the development of hearing loss, slow the progression of hearing loss, improve hearing, increase vestibular and/or cochlear hair cell numbers, increase vestibular and/or cochlear hair cell maturation, or increase vestibular and/or cochlear hair cell regeneration.

62. A kit comprising the nucleic acid vector of any one of claims 1-13 or the composition of claim 14.
